SEQUENCE LISTING (31 sequences)

SEQ ID NO: 1 - protein, 504 aa, Homo sapiens
MFPREKTWNISFAGCGFLGVYYVGVASCLREHAPFLVANATHIYGASAGALTATALVTGVCLGEAGAKFIEVSKEARKRFLGPLHPSFNLVKIIRSFLLKVLPADSHEHASGRLGISLTRVSDGENVIISHFNSKDELIQANVCSGFIPVYCGLIPPSLQGVRYVDGGISDNLPLYELKNTITVSPFSGESDICPQDSSTNIHELRVTNTSIQFNLRNLYRLSKALFPPEPLVLREMCKQGYRDGLRFLQRNGLLNRPNPLLALPPARPHGPEDKDQAVESAQAEDYSQLPGEDHILEHLPARLNEALLEACVEPTDLLTTLSNMLPVRLATAMMVPYTLPLESALSFTIRLLEWLPDVPEDIRWMKEQTGSICQYLVMRAKRKLGRHLPSRLPEQVELRRVQSLPSVPLSCAAYREALPGWMRNNLSLGDALAKWEECQRQLLLGLFCTNVAFPPEALRMRAPADPAPAPADPASPQHQLAGPAPLLSTPAPEARPVIGALGL

SEQ ID NO: 2 - DNA, 1515 bp, Homo sapiens
atgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgcccggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagctggccgggcctgcccccttgctgagcacccctgctcccgaggcccggcccgtgatcggggccctggggctgtga

SEQ ID NO: 3 - protein, 306 aa, Homo sapiens
MGGLTASDVHPTLGVQLFSAGIAACLADVITFPLDTAKVRLQVQGECPTSSVIRYKGVLGTITAVVKTEGRMKLYSGLPAGLQRQISSASLRIGLYDTVQEFLTAGKETPSLGSKILAGLTTGGVAVFIGQPTEVVKVRLQAQSHLHGIKPRYTGTYNAYRIIATTEGLTGLWKGTTPNLMRSVIINCTELVTYDLMKEAFVKNNILADDVPCHLVSALIAGFCATAMSSPVDVVKTRFINSPPGQYKSVPNCAMKVFTNEGPTAFFKGLVPSFLRLGSWNVIMFVCFEQLKRELSKSRQTMDCAT

SEQ ID NO: 4 - DNA, 921 bp, Homo sapiens
atggggggcctgacagcctcggacgtacacccgaccctgggggtccagctcttctcagctggaatagcggcgtgcttggcggacgtgatcaccttcccgctggacacggccaaagtccggctccaggtccaaggtgaatgcccgacgtccagtgttattaggtataaaggtgtcctgggaacaatcaccgctgtggtaaaaacagaagggcggatgaaactctacagcgggctgcctgcggggcttcagcggcaaatcagctccgcctctctcaggatcggcctctacgacacggtccaggagttcctcaccgcagggaaagaaacacctagtttaggaagcaagattttagctggtctaacgactggaggagtggcagtattcattgggcaacccacagaggtcgtgaaagtcagacttcaagcacagagccatctccacggaatcaaacctcgctacacggggacttataatgcgtacagaataatagcaacaaccgaaggcttgacgggtctttggaaagggactactcccaatctgatgagaagtgtcatcatcaattgtacagagctagtaacatatgatctaatgaaggaggcctttgtgaaaaacaacatattagcagatgacgtcccctgccacttggtgtcggctcttatcgctggattttgcgcaacagctatgtcctccccggtggatgtagtaaaaaccagatttattaattctccaccaggacagtacaaaagtgtgcccaactgtgcaatgaaagtgttcactaacgaaggaccaacggctttcttcaaggggttggtaccttccttcttgcgacttggatcctggaacgtcattatgtttgtgtgctttgaacaactgaaacgagaactgtcaaagtcaaggcagactatggactgtgccacataa

SEQ ID NO: 5 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 6 - DNA, 504 bp, Homo sapiens
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 7 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 8 - DNA, 735 bp, Homo sapiens
atgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 9 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 10 - DNA, 327 bp, Homo sapiens
atgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctga

SEQ ID NO: 11 - protein, 658 aa, Homo sapiens
MVPRLLLRAWPRGPAVGPGAPSRPLSAGSGPGQYLQRSIVPTMHYQDSLPRLPIPKLEDTIRRYLSAQKPLLNDGQFRKTEQFCKSFENGIGKELHEQLVALDKQNKHTSYISGPWFDMYLSARDSVVLNFNPFMAFNPDPKSEYNDQLTRATNMTVSAIRFLKTLRAGLLEPEVFHLNPAKSDTITFKRLIRFVPSSLSWYGAYLVNAYPLDMSQYFRLFNSTRLPKPSRDELFTDDKARHLLVLRKGNFYIFDVLDQDGNIVSPSEIQAHLKYILSDSSPAPEFPLAYLTSENRDIWAELRQKLMSSGNEESLRKVDSAVFCLCLDDFPIKDLVHLSHNMLHGDGTNRWFDKSFNLIIAKDGSTAVHFEHSWGDGVAVLRFFNEVFKDSTQTPAVTPQSQPATTDSTVTVQKLNFELTDALKTGITAAKEKFDATMKTLTIDCVQFQRGGKEFLKKQKLSPDAVAQLAFQMAFLRQYGQTVATYESCSTAAFKHGRTETIRPASVYTKRCSEAFVREPSRHSAGELQQMMVECSKYHGQLTKEAAMGQGFDRHLFALRHLAAAKGIILPELYLDPAYGQINHNVLSTSTLSSPAVNLGGFAPVVSDGFGVGYAVHDNWIGCNVSSYPGRNAREFLQCVEKALEDMFDALEGKSIKS

SEQ ID NO: 12 - DNA, 1977 bp, Homo sapiens
atggtgccccgcctgctgctgcgcgcctggccccggggccccgcggttggtccgggagcccccagtcggcccctcagcgccggctccgggcccggccagtacctgcagcgcagcatcgtgcccaccatgcactaccaggacagcctgcccaggctgcctattcccaaacttgaagacaccattaggagatacctcagtgcacagaagcctctcttgaatgatggccagttcaggaaaacagaacaattttgcaagagttttgaaaatgggattggaaaagaactgcatgagcagctggttgctctggacaaacagaataaacatacaagctacatttcgggaccctggtttgatatgtacctatctgctcgagactccgttgttctgaactttaatccatttatggctttcaatcctgacccaaaatctgagtataatgaccagctcacccgggcaaccaacatgactgtttctgccatccggtttctgaagacactccgggctggccttctggagccagaagtgttccacttgaaccctgcaaaaagtgacactatcaccttcaagagactcatacgctttgtgccttcctctctgtcctggtatggggcctacctggtcaatgcgtatcccctggatatgtcccagtattttcggcttttcaactcaactcgtttacccaaacccagtcgggatgaactcttcactgatgacaaggccagacacctcctggtcctaaggaaaggaaatttttatatctttgatgtcctggatcaagatgggaacattgtgagcccctcggaaatccaggcacatctgaagtacattctctcagacagcagccccgcccccgagtttcccctggcatacctgaccagtgagaaccgagacatctgggcagagctcaggcagaagctgatgagtagtggcaatgaggagagcctgaggaaagtggactcggcagtgttctgtctctgcctagatgacttccccattaaggaccttgtccacttgtcccacaatatgctgcatggggatggcacaaaccgctggtttgataaatcctttaacctcattatcgccaaggatggctctactgccgtccactttgagcactcttggggtgatggtgtggcagtgctcagattttttaatgaagtatttaaagacagcactcagacccctgccgtcactccacagagccagccagctaccactgactctactgtcacggtgcagaaactcaacttcgagctgactgatgccttaaagactggcatcacagctgctaaggaaaagtttgatgccaccatgaaaaccctcactattgactgcgtccagtttcagagaggaggcaaagaattcctgaagaagcaaaagctgagccctgacgcagttgcccagctggcattccagatggccttcctgcggcagtacgggcagacagtggccacctacgagtcctgtagcactgccgcattcaagcacggccgcactgagaccatccgcccggcctccgtctatacaaagaggtgctctgaggcctttgtcagggagccctccaggcacagtgctggtgagcttcagcagatgatggttgagtgctccaagtaccatggccagctgaccaaagaagcagcaatgggccagggctttgaccgacacttgtttgctctgcggcatctggcagcagccaaagggatcatcttgcctgagctctacctggaccctgcatacgggcagataaaccacaatgtcctgtccacgagcacactgagcagcccagcagtgaaccttgggggctttgcccctgtggtctctgatggctttggtgttgggtatgctgttcatgacaactggataggctgcaatgtctcttcctacccaggccgcaatgcccgggagtttctccaatgtgtggagaaggccttagaagacatgtttgatgccttagaaggcaaatccatcaaaagttaa

SEQ ID NO: 13 - protein, 420 aa, Homo sapiens
MAAGFGRCCRVLRSISRFHWRSQHTKANRQREPGLGFSFEFTEQQKEFQATARKFAREEIIPVAAEYDKTGEYPVPLIRRAWELGLMNTHIPENCGGLGLGTFDACLISEELAYGCTGVQTAIEGNSLGQMPIIIAGNDQQKKKYLGRMTEEPLMCAYCVTEPGAGSDVAGIKTKAEKKGDEYIINGQKMWITNGGKANWYFLLARSDPDPKAPANKAFTGFIVEADTPGIQIGRKELNMGQRCSDTRGIVFEDVKVPKENVLIGDGAGFKVAMGAFDKTRPVVAAGAVGLAQRALDEATKYALERKTFGKLLVEHQAISFMLAEMAMKVELARMSYQRAAWEVDSGRRNTYYASIAKAFAGDIANQLATDAVQILGGNGFNTEYPVEKLMRDAKIYQIYEGTSQIQRLIVAREHIDKYK

SEQ ID NO: 14 - DNA, 1266 bp, Homo sapiens
atggcagcggggttcgggcgatgctgcagggtcctgagaagtatttctcgttttcattggagatcacagcatacaaaagccaatcgacaacgtgaaccaggattaggatttagttttgagttcaccgaacagcagaaagaatttcaagctactgctcgtaaatttgccagagaggaaatcatcccagtggctgcagaatatgataaaactggtgaatatccagtccccctaattagaagagcctgggaacttggtttaatgaacacacacattccagagaactgtggaggtcttggacttggaacttttgatgcttgtttaattagtgaagaattggcttatggatgtacaggggttcagactgctattgaaggaaattctttggggcaaatgcctattattattgctggaaatgatcaacaaaagaagaagtatttggggagaatgactgaggagccattgatgtgtgcttattgtgtaacagaacctggagcaggctctgatgtagctggtataaagaccaaagcagaaaagaaaggagatgagtatattattaatggtcagaagatgtggataaccaacggaggaaaagctaattggtattttttattggcacgttctgatccagatcctaaagctcctgctaataaagcctttactggattcattgtggaagcagataccccaggaattcagattgggagaaaggaattaaacatgggccagcgatgttcagatactagaggaattgtcttcgaagatgtgaaagtgcctaaagaaaatgttttaattggtgacggagctggtttcaaagttgcaatgggagcttttgataaaaccagacctgtagtagctgctggtgctgttggattagcacaaagagctttggatgaagctaccaagtatgccctggaaaggaaaactttcggaaagctacttgtagagcaccaagcaatatcatttatgctggctgaaatggcaatgaaagttgaactagctagaatgagttaccagagagcagcttgggaggttgattctggtcgtcgaaatacctattatgcttctattgcaaaggcatttgctggagatattgcaaatcagttagctactgatgctgtgcagatacttggaggcaatggatttaatacagaatatcctgtagaaaaactaatgagggatgccaaaatctatcagatttatgaaggtacttcacaaattcaaagacttattgtagcccgtgaacacattgacaagtacaaaaattaa

SEQ ID NO: 15 - protein, 1276 aa, Homo sapiens
MRSKARARKLAKSDGDVVNNMYEPNRDLLASHSAEDEAEDSAMSPIPVGSPPPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQVIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNFIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPYRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQAYAMMLSLSEDTPLHTPSQGSLDAWLKVTGATSESGAFHPINHL

SEQ ID NO: 16 - DNA, 3831 bp, Homo sapiens
atgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggtcaccgccccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcaggtaatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacttcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctacaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccaggcatatgcaatgatgctgtccctttccgaagacactcctctccacaccccctcccagggttctctggacgcttggttgaaggtcactggagccacgtcggagtctggagcatttcaccccatcaaccacctctga

SEQ ID NO: 17 - protein, 358 aa, Homo sapiens
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 18 - DNA, 1077 bp, Homo sapiens
atggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtga

SEQ ID NO: 19 - protein, 11 aa, Artificial Sequence
  Synthetic peptide
YGRKKRRQRRR

SEQ ID NO: 20 - protein, 12 aa, Artificial Sequence
  Synthetic peptide
RRQRRTSKLMKR

SEQ ID NO: 21 - protein, 27 aa, Artificial Sequence
  Synthetic peptide
GWTLNSAGYLLGKINLKALAALAKKIL

SEQ ID NO: 22 - protein, 33 aa, Artificial Sequence
  Synthetic peptide
KALAWEAKLAKALAKALAKHLAKALAKALKCEA

SEQ ID NO: 23 - protein, 16 aa, Artificial Sequence
  Synthetic peptide
RQIKIWFQNRRMKWKK

SEQ ID NO: 24 - protein, 9 aa, Artificial Sequence
  Synthetic peptide
RKKRRQRRR

SEQ ID NO: 25 - protein, 8 aa, Artificial Sequence
  Synthetic peptide
RKKRRQRR

SEQ ID NO: 26 - protein, 11 aa, Artificial Sequence
  Synthetic peptide
YARAAARQARA

SEQ ID NO: 27 - protein, 11 aa, Artificial Sequence
  Synthetic peptide
THRLPRRRRRR

SEQ ID NO: 28 - protein, 11 aa, Artificial Sequence
  Synthetic peptide
GGRRARRRRRR

SEQ ID NO: 29 - DNA, 22 bp, Artificial Sequence
  Synthetic oligonucleotide
agcttgctgtcactcctctaca

SEQ ID NO: 30 - DNA, 20 bp, Artificial Sequence
  Synthetic oligonucleotide
tgaggaaagggttgaccttg

SEQ ID NO: 31 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

(19) United States
(12) Patent Application Publication
(10) Pub. No.: US 2014/0147431 A1
(43) Pub. Date: May 29, 2014

(54) Compositions and Methods for Modulating Desnutrin-Mediated Adipocyte Lipolysis

(71) Applicants: Hei Sook Sul, Berkeley, CA (US); Maryam Ahmadian, Berkeley, CA (US)

(72) Inventors: Hei Sook Sul, Berkeley, CA (US); Maryam Ahmadian, Berkeley, CA (US)

(73) Assignee: The Regents of the University of California, Oakland, CA (US)

(21) Appl. No.: 13/825,086

(22) PCT Filed: Sep. 16, 2011
(86) PCT No.: PCT/US11/51991

§ 371 Date: Nov. 4, 2013

Related U.S. Application Data
(60) Provisional application No. 61/384,617, filed on Sep. 20, 2010.

(51) Int. Cl. A61K 38/46

(52) U.S. Cl. CPC A61K 38/465
USPC 424/94.6; 514/44 R; 435/375; 435/455; 435/18

(57) ABSTRACT

The present disclosure provides methods of converting white adipose tissue to brown adipose tissue in an individual, generally involving modulating desnutrin-mediated lipolysis in adipocytes in the individual. The present disclosure further provides methods for treating obesity. The present disclosure further provides methods of identifying an agent that increases the level and/or activity of desnutrin in an adipocyte.

### CROSS-REFERENCE

This application claims the benefit of U.S. Provisional Patent Application No. 61/384,617, filed Sep. 20, 2010, which application is incorporated herein by reference in its entirety.

### STATEMENT REGARDING FEDERALLY SPONSORED RESEARCH

This invention was made with government support under Grant No. R01 DK075682 awarded by the National Institutes of Health. The government has certain rights in the invention.

### BACKGROUND

Adipose tissue plays a critical role in controlling whole-body energy balance. As the primary fuel reserve in mammals, white adipose tissue (WAT) has the unique function of storing triacylglycerol (TAG) during times of energy surplus and hydrolyzing TAG (lipolysis) during times of energy deprivation to provide fatty acids (FAs) as fuel for other organs. Brown adipose tissue (BAT), on the other hand, is specialized in thermogenesis, using FAs generated through lipolysis to activate UCP-1 and as substrates for mitochondrial 13-oxidation. These two tissues can be distinguished from each other based on their morphology, gene expression profile as well as by characteristic biochemical functions. Nevertheless, lipolysis is a critical metabolic process in both WAT and BAT. Lipolysis occurs in three stages with different enzymes acting at each step: TAG is sequentially hydrolyzed to form diacylglycerol (DAG), by desnutrin/ATGL/iPLA₂ζ (gene name: PNPLA2, TTS2.2) which has been identified as the major TAG hydrolase in adipose tissue, but is also expressed in other tissues. DAG is then hydrolyzed by hormone-sensitive lipase (HSL) to monoacylglycerol (MAG), and subsequently glycerol, with a FA released at each stage.

### LITERATURE

Villena et al. (2004) J. Biol. Chem. 279:47066; Ahmadian et al. (2009) Diabetes 58:855; Frühbeck et al. (2009) Trends Pharmacol. Sci. 30:387; Tiraby et al. (2003) J. Biol. Chem. 278:33370; Jenkins et al. (2004) J. Biol. Chem. 279:48968.

### SUMMARY OF THE INVENTION

The present disclosure provides methods of converting white adipose tissue to brown adipose tissue in an individual, generally involving modulating desnutrin-mediated lipolysis in adipocytes in the individual. The present disclosure further provides methods for treating obesity. The present disclosure further provides methods of identifying an agent that increases the level and/or activity of desnutrin in an adipocyte.

### BRIEF DESCRIPTION OF THE DRAWINGS

FIGS. 1A-G depict increased adiposity in desnutrin-ASKO mice.

FIGS. 2A-G depict the effect of decreased lipolysis in desnutrin-ASKO mice on thermogenesis and energy expenditure.

FIGS. 3A-N depict the effect of desnutrin ablation on conversion of BAT to WAT, and the effect of phosphorylation of desnutrin by 5′-adenosine monophosphate kinase (AMPK) on lipolysis.

FIGS. 4A-F depict improved insulin sensitivity in desnutrin-ASKO mice.

FIG. 5 provides an amino acid sequence of a human desnutrin (PNPLA2) polypeptide.

FIG. 6 provides a nucleotide sequence encoding a human desnutrin polypeptide.

FIG. 7 provides an amino acid sequence of a human Ucp1 polypeptide.

FIG. 8 provides a nucleotide sequence encoding a human Ucp1 polypeptide.

FIG. 9 provides an amino acid sequence of a human leptin polypeptide.

FIG. 10 provides a nucleotide sequence encoding a human leptin polypeptide.

FIG. 11 provides an amino acid sequence of a human adiponectin polypeptide.

FIG. 12 provides a nucleotide sequence encoding a human adiponectin polypeptide.

FIG. 13 provides an amino acid sequence of a human resistin polypeptide.

FIG. 14 provides a nucleotide sequence encoding a human resistin polypeptide.

FIG. 15 provides an amino acid sequence of a human CPT1 polypeptide.

FIGS. 16A and 16B provide a nucleotide sequence encoding a human CPT1 polypeptide.

FIG. 17 provides an amino acid sequence of a human MCAD polypeptide.

FIG. 18 provides a nucleotide sequence encoding a human MCAD polypeptide.

FIG. 19 provides an amino acid sequence of a human PRDM16 polypeptide.

FIGS. 20A-C provide a nucleotide sequence encoding a human PRDM16 polypeptide.

FIG. 21 provides an amino acid sequence of a human CEBPα polypeptide.

FIG. 22 provides a nucleotide sequence encoding a human CEBPα polypeptide.

### DEFINITIONS

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “nucleic acid” and “polynucleotide” are used interchangeably herein and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers.

The term “operably linked” refers to functional linkage between molecules to provide a desired function. For example, “operably linked” in the context of nucleic acids refers to a functional linkage between nucleic acids to provide a desired function such as transcription, translation, and the like, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide.

A “host cell,” as used herein, denotes an in vivo or in vitro cell (e.g., a eukaryotic cell cultured as a unicellular entity), which eukaryotic cell can be, or has been, used as recipients for a nucleic acid (e.g., an exogenous nucleic acid) or an exogenous polypeptide(s), and include the progeny of the original cell which has been modified by introduction of the exogenous polypeptide(s) or genetically modified by the nucleic acid. It is understood that the progeny of a single cell may not necessarily be completely identical in morphology or in genomic or total DNA complement as the original parent, due to natural, accidental, or deliberate mutation.

The term “genetic modification” and refers to a permanent or transient genetic change induced in a cell following introduction of new nucleic acid (i.e., nucleic acid exogenous to the cell). Genetic change (“modification”) can be accomplished by incorporation of the new nucleic acid into the genome of the host cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

As used herein, the term “exogenous nucleic acid” refers to a nucleic acid that is not normally or naturally found in and/or produced by a cell in nature, and/or that is introduced into the cell (e.g., by electroporation, transfection, infection, lipofection, or any other means of introducing a nucleic acid into a cell).

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

A “therapeutically effective amount” or “efficacious amount,” in the context of increasing BAT relative to WAT, refers to the amount of a polypeptide or nucleic acid that, when administered to a mammal or other subject, is sufficient to effect an increase in BAT relative to WAT in the mammal. A “therapeutically effective amount” or “efficacious amount,” in the context of treating a disease such as obesity, refers to the amount of a polypeptide or nucleic acid that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an adipocyte” includes a plurality of such adipocytes and reference to “the desnutrin polypeptide” includes reference to one or more desnutrin polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of increasing the amount of brown adipose tissue relative to white adipose tissue in an individual, generally involving modulating desnutrin-mediated lipolysis in adipocytes in the individual. The present disclosure provides methods of converting white adipose tissue to brown adipose tissue in an individual, generally involving modulating desnutrin-mediated lipolysis in adipocytes in the individual. The present disclosure further provides methods of identifying an agent that increases the level and/or activity of desnutrin in an adipocyte.

### Methods of Increasing Brown Adipose Tissue Relative to White Adipose Tissue

The present disclosure provides methods of increasing the amount of brown adipose tissue relative to white adipose tissue in an individual, generally involving modulating desnutrin-mediated lipolysis in adipocytes in the individual. The present disclosure provides methods of converting white adipose tissue to brown adipose tissue in an individual, generally involving modulating desnutrin-mediated lipolysis in adipocytes in the individual.

In some embodiments, a subject method involves contacting an adipocyte of WAT with an effective amount of an agent that activates desnutrin. Desnutrin can be activated by AMPK, which phosphorylates S406 on desnutrin. An agent that activates AMPK can increase conversion of WAT to BAT. Agents that activate AMPK include, e.g., 5-amino-4-imidazolecarboxamide riboside (AICAR), metformin, phenformin, and the like. Agents that activate AMPK also include compounds disclosed in WO 08/006,432; WO 05/051298; WO 05/020892; thiazole derivatives disclosed in US 2007/0015665; pyrazole compounds disclosed in US 2007/0032529; thienopyridones disclosed in US 2006/0287356; thienopyridone compounds disclosed in US 2005/0038068; and cyclic benzimidazole compound disclosed in US 2011/0218174. In some embodiments, the contacting is carried out in vitro. In some embodiments, the contacting is carried out ex vivo. In some embodiments, the contacting is carried out in vivo. In some embodiments, a WAT adipocyte is contacted with an effective amount of AICAR, metformin, a thiazole compound disclosed in US 2007/0015665, a pyrazole compound disclosed in US 2007/0032529, a thienopyridone disclosed in US 2006/0287356, a thienopyridone compounds disclosed in US 2005/0038068, or a cyclic benzimidazole compound disclosed in US 2011/0218174.

An effective amount of an agent that activates AMPK is an amount that, when contacted with a WAT adipocyte, results in conversion of at least 5%, at least 10%, at least 15%, at least 20%, or more than 20%, of the adipocytes in WAT to brown adipocytes. In some embodiments, a subject method results in conversion of at least 25%, 30%, 35%, 40%, 44%, 50%, 57%, 62%, 70%, 74%, 75%, 80%, 90%, or other percent of cells greater than 5%, of the adipocytes in WAT to brown adipocytes.

In some embodiments, a subject method generally involves contacting an adipocyte (e.g., an adipocyte of WAT) with a desnutrin polypeptide or a nucleic acid comprising a nucleotide sequence encoding a desnutrin polypeptide, where the desnutrin polypeptide or the nucleic acid comprising a nucleotide sequence encoding a desnutrin polypeptide enters the adipocyte, resulting in a level of desnutrin in the adipocyte that is higher than the level of endogenous desnutrin in the adipocyte. For example, an adipocyte is contacted with a nucleic acid comprising a nucleotide sequence encoding a desnutrin polypeptide, the nucleic acid enters the adipocyte, and the encoded desnutrin is produced in the adipocyte. In some embodiments, the contacting is carried out in vitro. In some embodiments, the contacting is carried out ex vivo. In some embodiments, the contacting is carried out in vivo.

In some embodiments, a subject method increases the level of desnutrin in an adipocyte such that the level of desnutrin in the adipocyte is at least about 10%, at least about 20%, at least about 25%, at least about 30%, at least about 40%, at least about 50%, at least about 60%, at least about 70%, at least about 80%, at least about 90%, at least about 2-fold, at least about 2.5-fold, at least about 3-fold, at least about 4-fold, at least about 5-fold, at least about 10-fold, or more than 10-fold, higher than the endogenous level of desnutrin in the adipocyte, e.g., higher than the level of desnutrin in the adipocyte before contacting with the desnutrin polypeptide, or with the nucleic acid comprising a nucleotide sequence encoding a desnutrin polypeptide.

In some embodiments, a subject method increases the proportion of a population of WAT adipocytes that are converted to BAT adipocytes. For example, in some embodiments, a subject method results in conversion of at least 5%, at least 10%, at least 15%, at least 20%, or more than 20%, of the adipocytes in WAT to brown adipocytes. In some embodiments, a subject method results in conversion of at least 25%, 30%, 35%, 40%, 44%, 50%, 57%, 62%, 70%, 74%, 75%, 80%, 90%, or other percent of cells greater than 5%, of the adipocytes in WAT to brown adipocytes.

In some embodiments, a subject method results in increased expression of gene products that are markers of BAT. For example, brown adipocytes (cells of BAT) have higher levels of Ucp1, CEBP alpha/beta, PPARα, CPT1β, Cidea, PRDM16, and glycerol kinase, compared to white adipocytes (cells of WAT). In some embodiments, contacting an adipocyte with a desnutrin polypeptide, or a nucleic acid comprising a nucleotide sequence encoding a desnutrin polypeptide, increases the level of a BAT-selective gene product (mRNA and/or polypeptide) in the adipocyte by at least about 10%, at least about 20%, at least about 25%, at least about 30%, at least about 40%, at least about 50%, at least about 60%, at least about 70%, at least about 80%, at least about 90%, at least about 2-fold, at least about 2.5-fold, at least about 3-fold, at least about 4-fold, at least about 5-fold, at least about 10-fold, or more than 10-fold, compared to the level of the BAT-selective gene product in the adipocyte before contacting with the desnutrin polypeptide, or with the nucleic acid comprising a nucleotide sequence encoding a desnutrin polypeptide. An example of a gene product whose expression in BAT is increased by desnutrin is peroxisome proliferator-activated receptor-alpha (PPAR α). See, e.g., GenBank Accession No. NP_{—}001001928; Cronet et al. (2001) Structure 9:699; and SEQ ID NO:31.

In some embodiments, a subject method results in an increase in uncoupling or fatty acid oxidation in a WAT adipocyte by at least about 10%, at least about 20%, at least about 25%, at least about 30%, at least about 40%, at least about 50%, at least about 60%, at least about 70%, at least about 80%, at least about 90%, at least about 2-fold, at least about 2.5-fold, at least about 3-fold, at least about 4-fold, at least about 5-fold, at least about 10-fold, or more than 10-fold, compared to the level of uncoupling or fatty acid oxidation in the adipocyte before contacting with the desnutrin polypeptide, or with the nucleic acid comprising a nucleotide sequence encoding a desnutrin polypeptide.

In some embodiments, contacting an adipocyte with a desnutrin polypeptide, or a nucleic acid comprising a nucleotide sequence encoding a desnutrin polypeptide, increases the level of uncoupling protein-1 (Ucp1) in the adipocyte by at least about 10%, at least about 20%, at least about 25%, at least about 30%, at least about 40%, at least about 50%, at least about 60%, at least about 70%, at least about 80%, at least about 90%, at least about 2-fold, at least about 2.5-fold, at least about 3-fold, at least about 4-fold, at least about 5-fold, at least about 10-fold, or more than 10-fold, compared to the level of Ucp1 in the adipocyte before contacting with the desnutrin polypeptide, or with the nucleic acid comprising a nucleotide sequence encoding a desnutrin polypeptide.

In some embodiments, a subject method results in conversion of WAT to BAT. Thus, e.g., in some embodiments, a subject method results in an increase in the level of BAT-selective gene products in an adipocyte, and results in a decrease in the level of WAT-selective gene products in the adipocyte. WAT-selective gene products include medium chain acyl-coenzyme A dehydrogenase (MCAD), RIP140, Igfbp3, DPT, Hoxc9, Tcf21, resistin, adiponectin, and leptin. For example, in some embodiments, a subject method results in an increase in the level of a BAT-selective gene product in an adipocyte, and results in a decrease of at least about 10%, at least about 20%, at least about 25%, at least about 30%, at least about 40%, at least about 50%, at least about 60%, at least about 70%, at least about 80%, or more than 80%, of a WAT-selective gene product in the adipocyte, compared to the level of the WAT-selective gene product in an adipocyte before contacting with a desnutrin polypeptide, or with a nucleic acid comprising a nucleotide sequence encoding a desnutrin polypeptide.

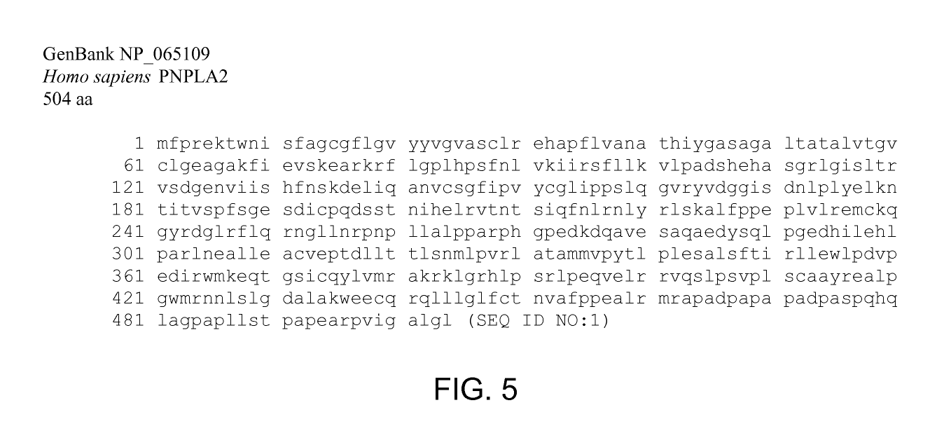

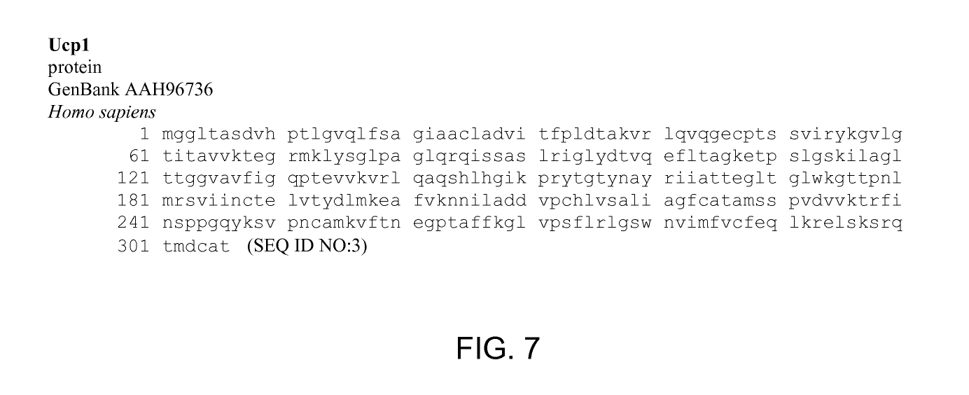

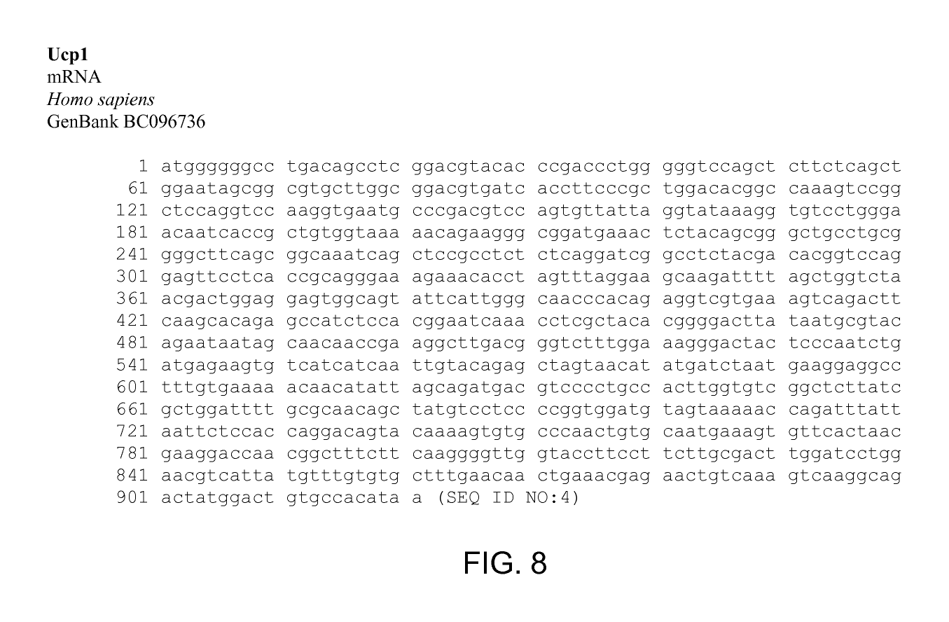

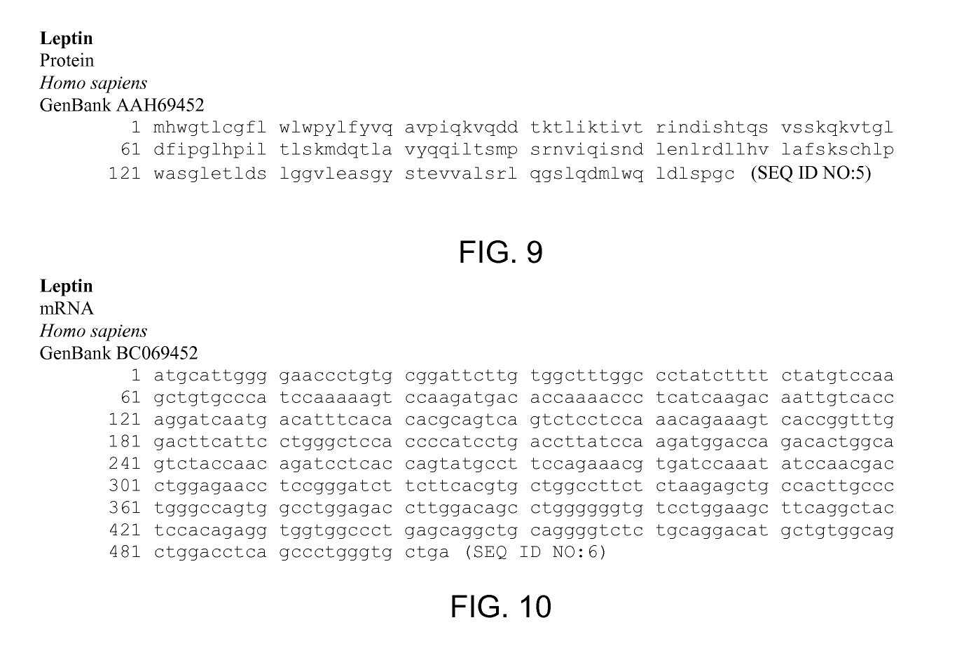

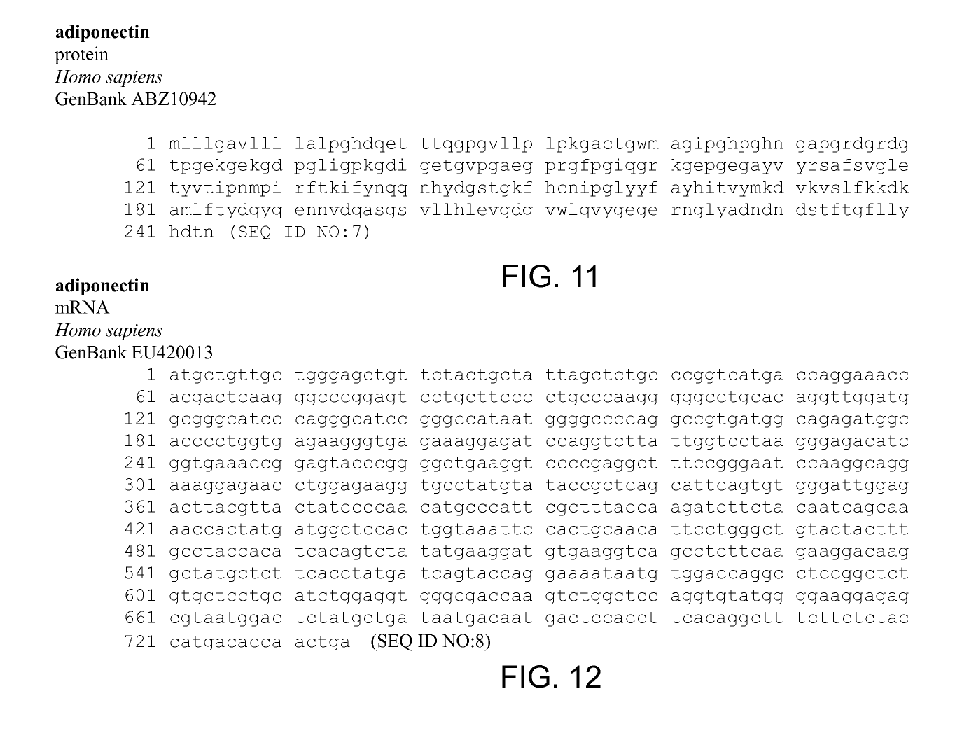

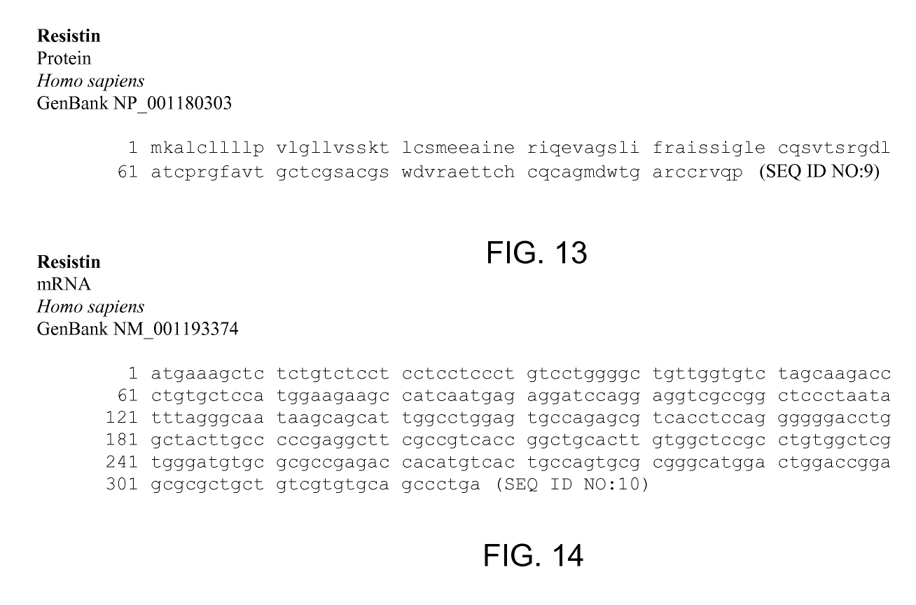

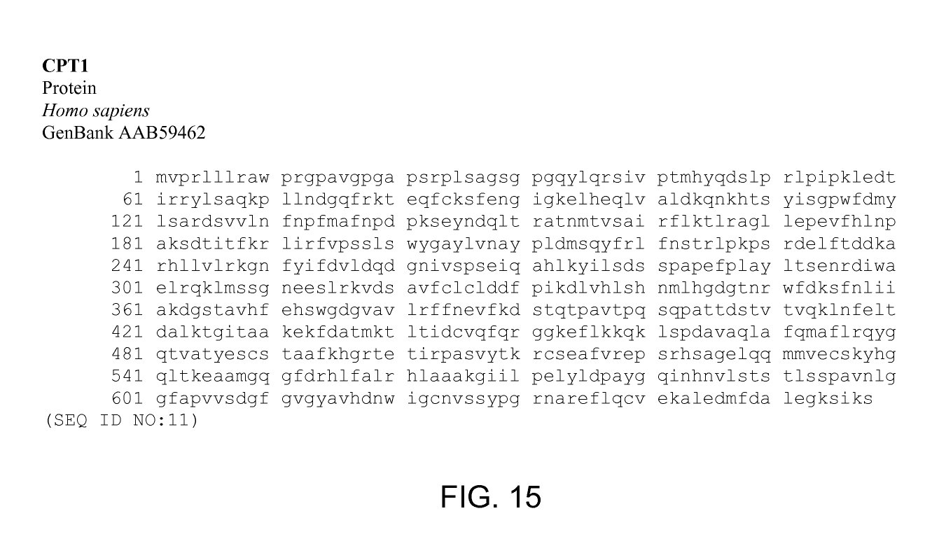

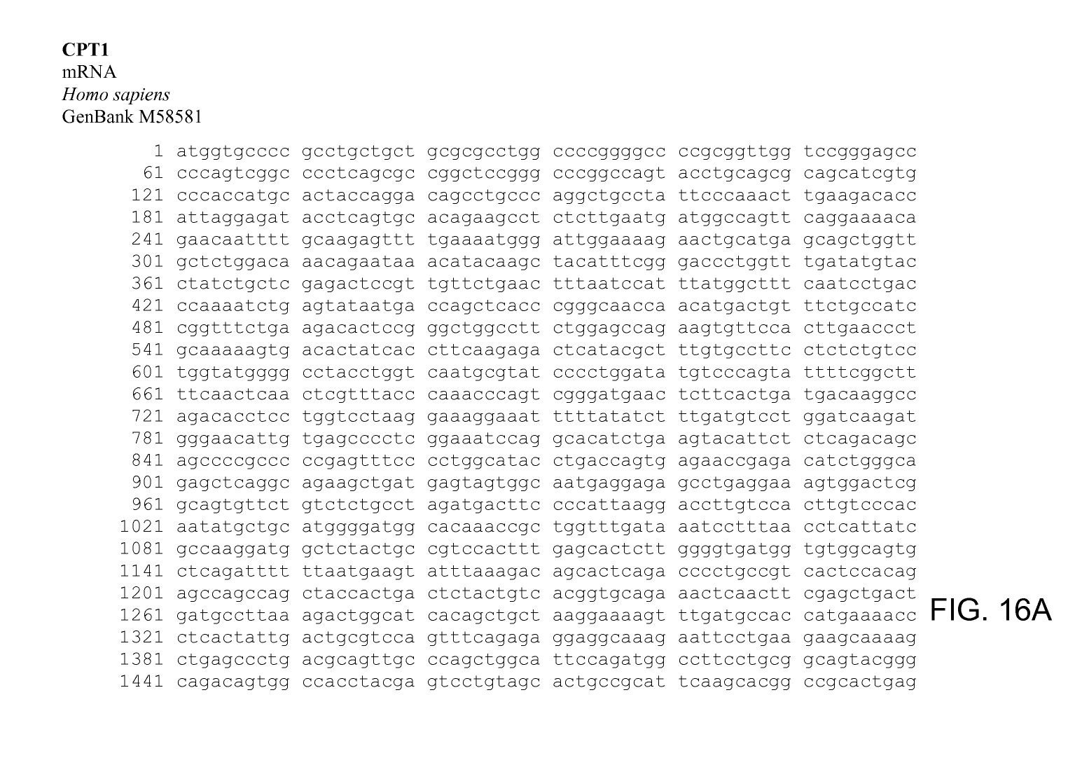

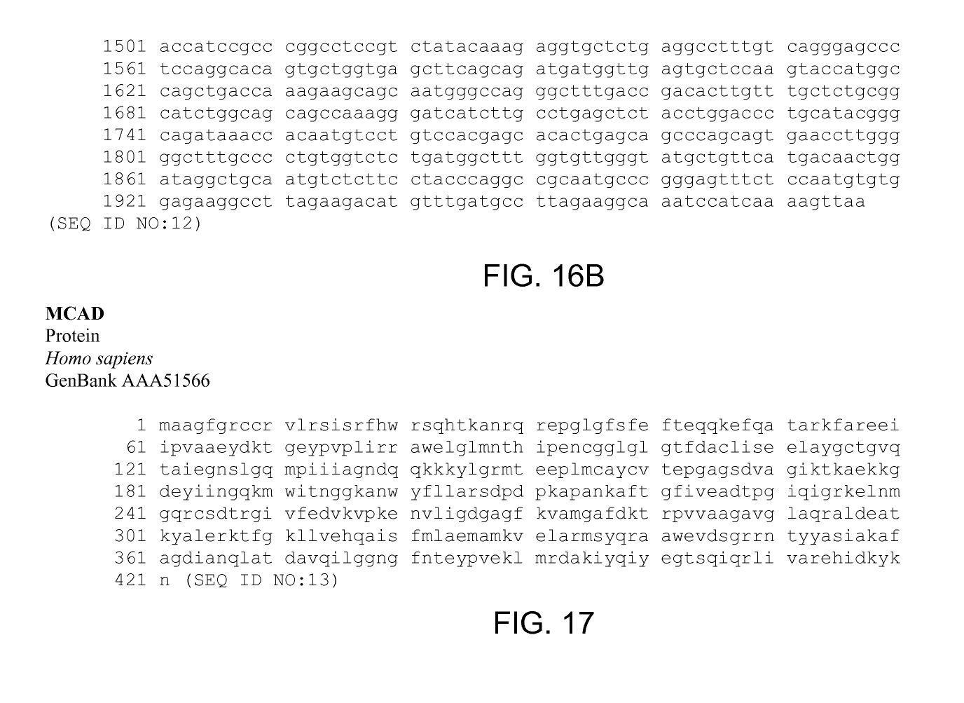

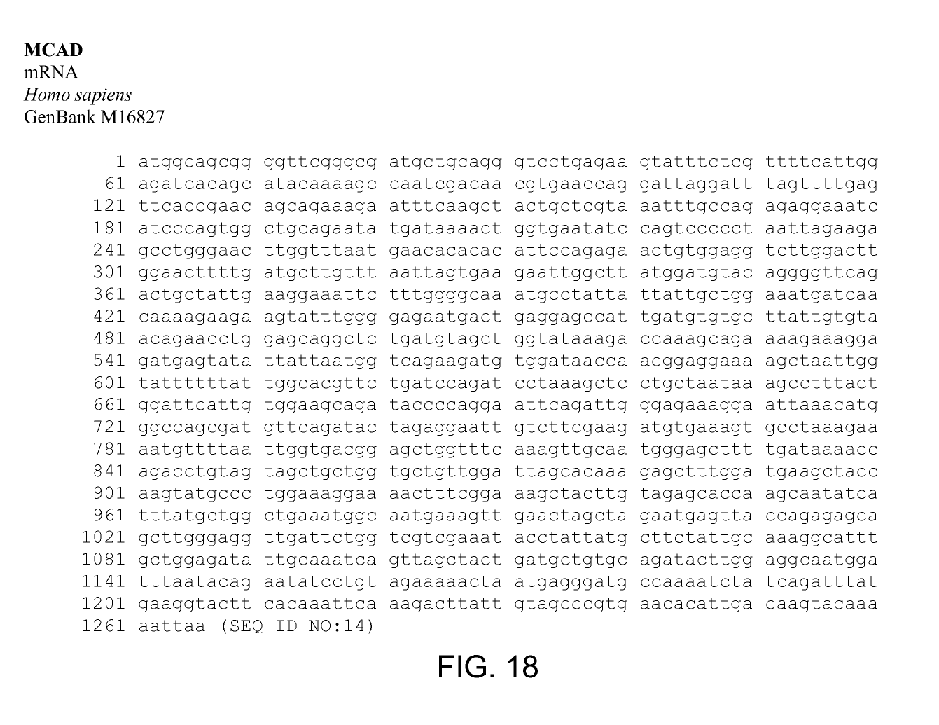

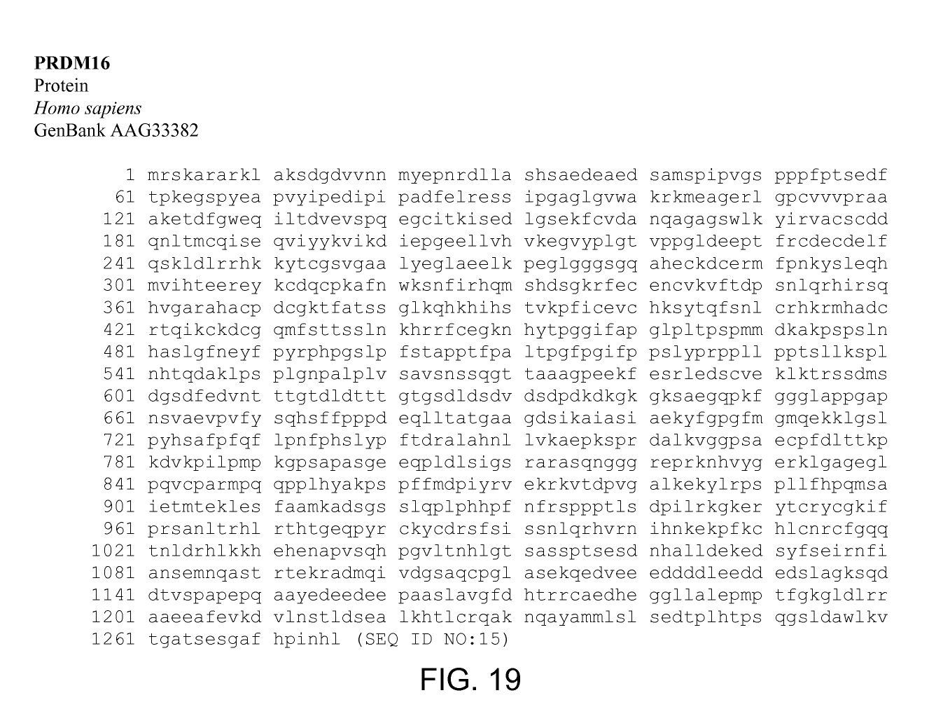

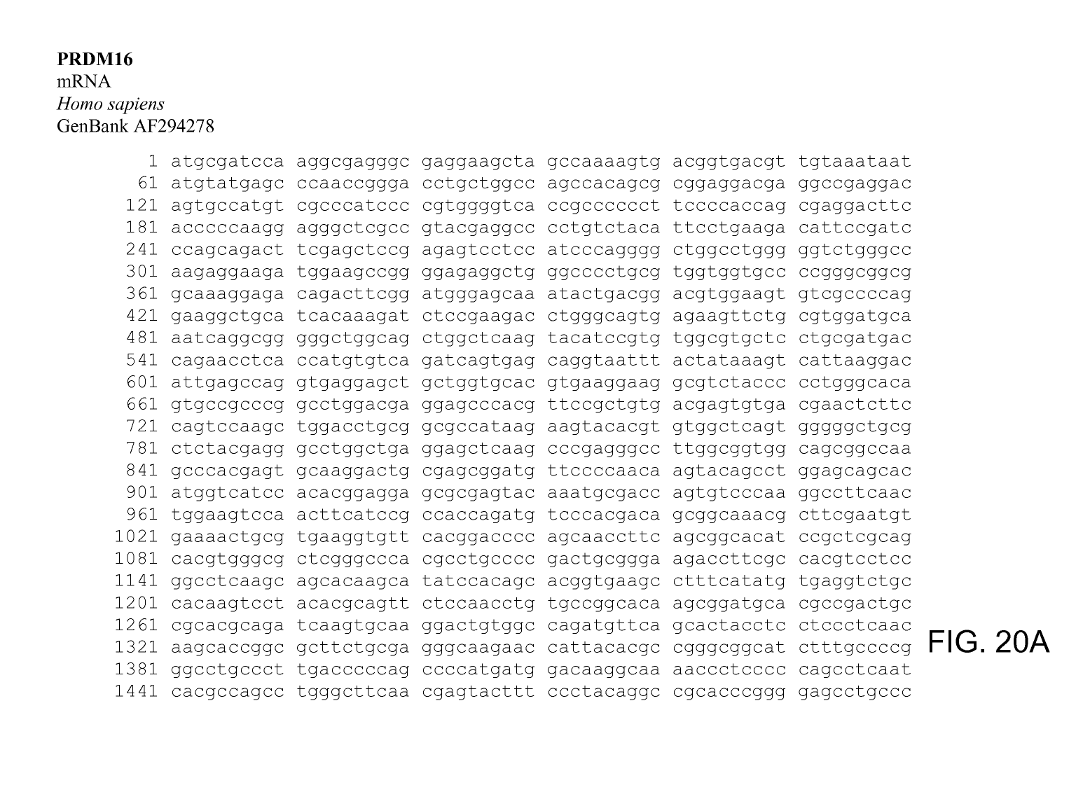

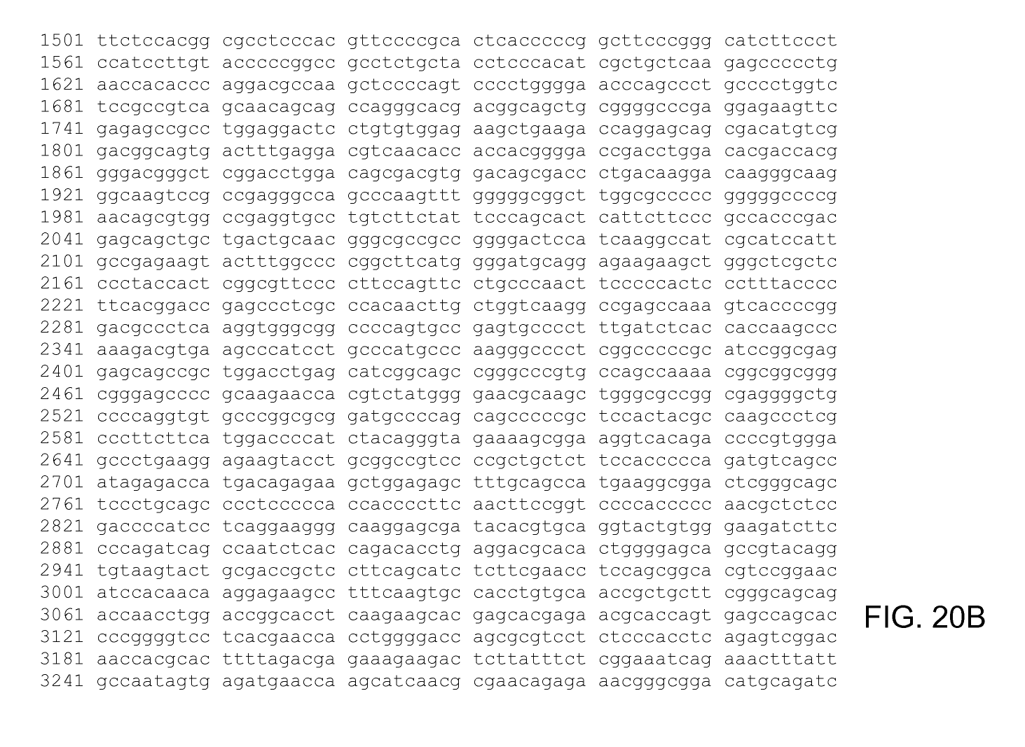

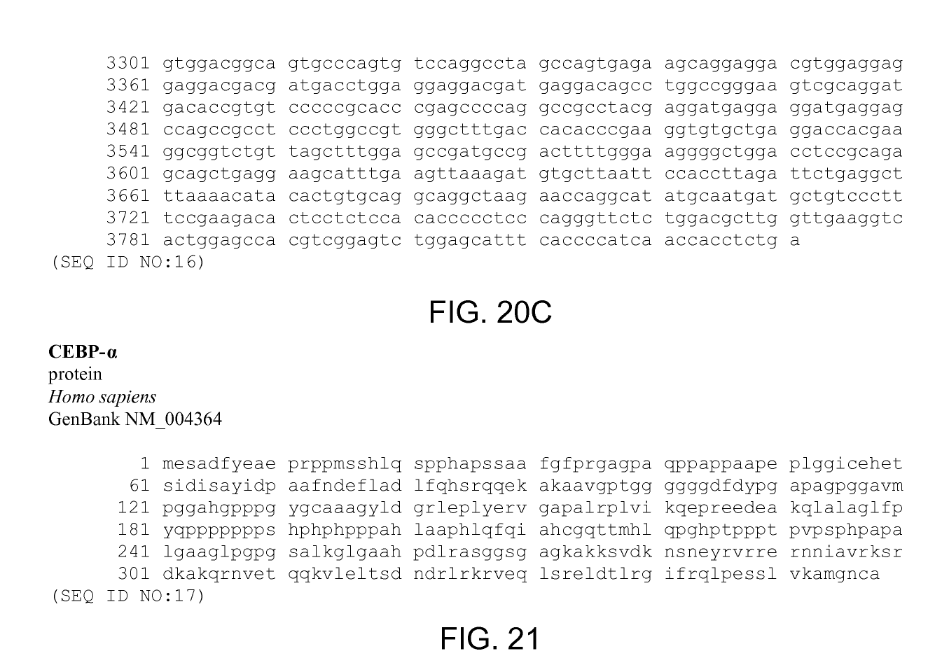

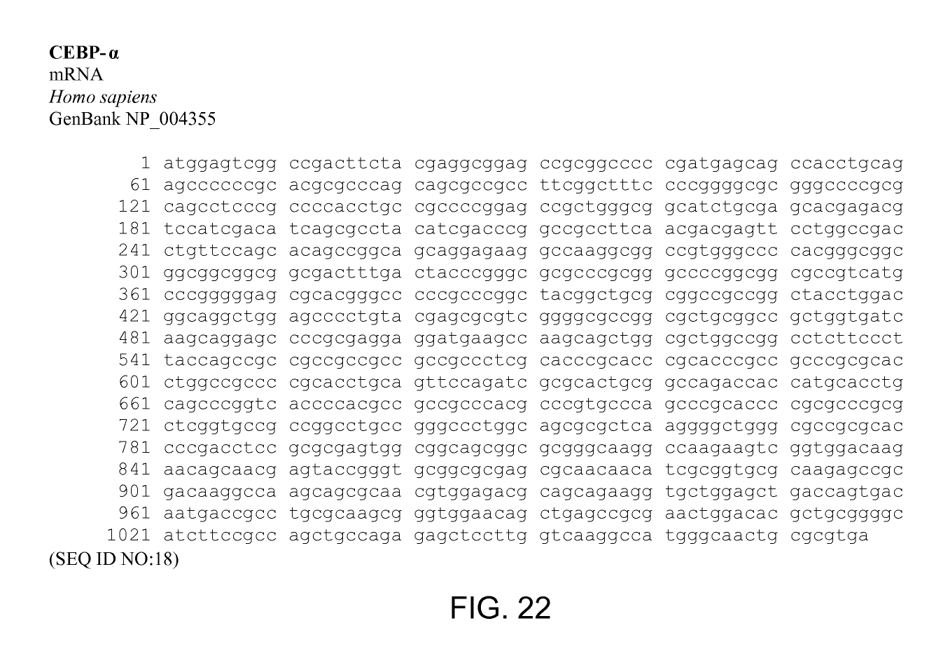

The expression of various markers specific to brown adipocytes or white adipocytes is detected by conventional biochemical or immunochemical methods (e.g., enzyme-linked immunosorbent assay; immunohistochemical assay; and the like). Alternatively, expression of nucleic acid encoding a BAT adipocyte-selective or WAT adipocyte-selective marker can be assessed. Expression of WAT-selective or BAT-selective marker-encoding nucleic acids in a cell can be confirmed by reverse transcriptase polymerase chain reaction (RT-PCR) or hybridization analysis, molecular biological methods which are commonly used for amplifying, detecting and analyzing mRNA coding for marker proteins. Nucleotide sequences of WAT-selective and BAT-selective marker-encoding nucleic acids are known and are available through public data bases such as GenBank; thus, marker-selective sequences for use as primers or probes are easily determined. FIGS. 5-22 provide amino acid sequences and nucleotide sequences of various BAT- and WAT-selective markers.

White adipocytes can also be distinguished from brown adipocytes histologically. White adipocytes have a scant ring of cytoplasm surrounding a single large lipid droplet; and their nuclei are flattened and eccentric within the cell. Brown adipocytes are polygonal in shape, have a considerable volume of cytoplasm and contain multiple lipid droplets of varying size; and their nuclei are round and almost centrally located. The mitochondria also differ between the two depots. Brown adipocytes have numerous round mitochondria with transverse cristae, whereas mitochondria from white adipocytes are less numerous and elongated with randomly oriented cristae. Thus, whether a subject method increases the level of BAT (e.g., increases the ratio of BAT to WAT) in an individual can be determined by examining cells from the individual histologically.

Desnutrin (also known as “patatin-like phospholipase domain containing 2” or PNPLA2, “ATGL,” “iPLA₂ζ,” “adipose triglyceride lipase,” “triglyceride hydrolyase,” “TTS2.2,” and “calcium-independent phospholipase A2”) catalyzes the conversion of triacylglycerides to diacylglycerides. Amino acid sequences of desnutrin polypeptides are known in the art. See, e.g., GenBank Accession No. NP_{—}065109 (Homo sapiens desnutrin); GenBank Accession Nos. NP_{—}001157161, NP_{—}080078, and AAH64747 (Mus musculus desnutrin); GenBank Accession Nos. NP_{—}001101979 and XP_{—}341961 (Rattus norvegicus desnutrin); GenBank Accession No. NP_{—}001039470 (Bos taurus desnutrin); and GenBank Accession No. XP_{—}854164 (Canis familiaris desnutrin). In some embodiments, a desnutrin polypeptide comprises an amino acid sequence having at least about 75%, at least about 80%, at least about 85%, at least about 90%, at least about 91%, at least about 92%, at least about 93%, at least about 94%, at least about 95%, at least about 98%, at least about 99%, or 100%, amino acid sequence identity to a contiguous stretch of from about 400 amino acids to about 450 amino acids, or from about 450 amino acids to about 504 amino acids, of the amino acid sequence depicted in FIG. 5.

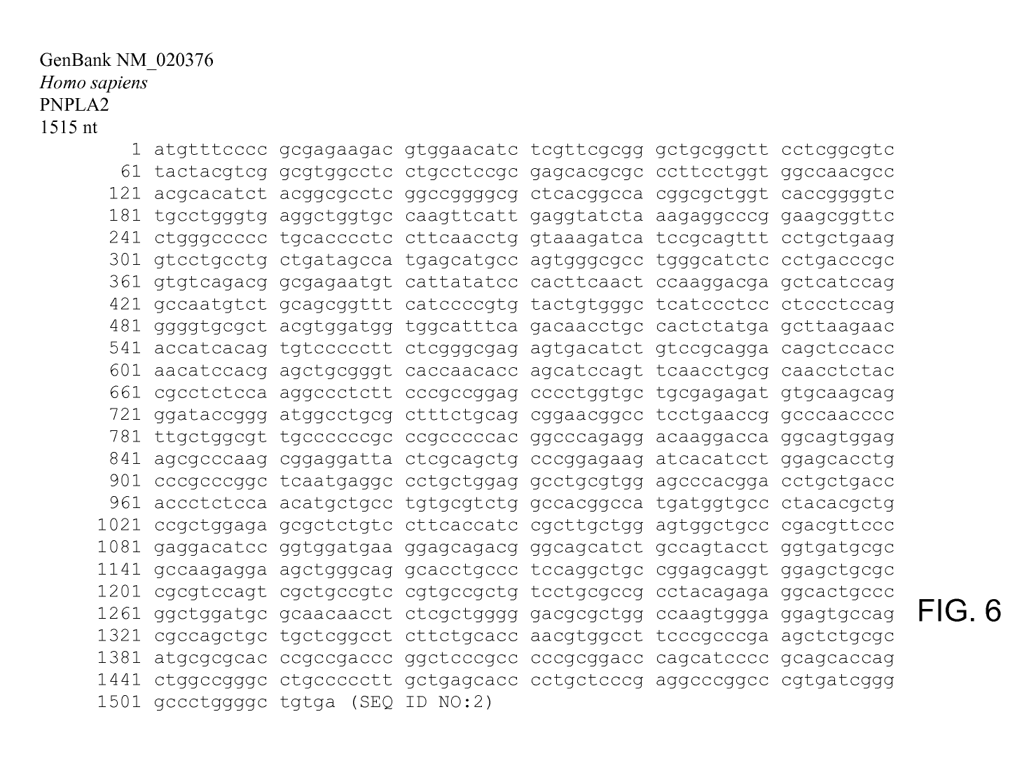

Nucleotide sequences encoding desnutrin polypeptides are also known in the art. See, e.g., GenBank Accession No. NM_{—}020376 (Homo sapiens desnutrin-encoding nucleotide sequence); GenBank Accession No. NM_{—}025802 (Mus musculus desnutrin-encoding nucleotide sequence); GenBank Accession No. XM_{—}341960 (Rattus norvegicus desnutrin-encoding nucleotide sequence); GenBank Accession No. NM_{—}001046005 (Bos taurus desnutrin-encoding nucleotide sequence); and GenBank Accession No. XM_{—}84907 (Canis familiaris desnutrin-encoding nucleotide sequence). In some embodiments, a desnutrin-encoding nucleotide sequence comprises a nucleotide sequence having at least about 75%, at least about 80%, at least about 85%, at least about 90%, at least about 91%, at least about 92%, at least about 93%, at least about 94%, at least about 95%, at least about 98%, at least about 99%, or 100%, nucleotide sequence identity to a contiguous stretch of from about 1450 nucleotides to about 1500 nucleotides, or from about 1500 nucleotides to 1515 nucleotides, of the nucleotide sequence depicted in FIG. 6.

Introduction of Exogenous Desnutrin Polypeptide into an Adipocyte

In some embodiments, introduction of exogenous desnutrin polypeptide into an adipocyte is achieved by contacting the adipocyte with an exogenous desnutrin polypeptide, such that the exogenous desnutrin polypeptide is taken up into the adipocyte.

In some embodiments, an exogenous desnutrin polypeptide comprises a protein transduction domain, which facilitates entry of the exogenous desnutrin polypeptide into a cell. “Protein Transduction Domain” or PTD refers to a polypeptide, polynucleotide, carbohydrate, or organic or inorganic compound that facilitates traversal of a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an exogenous desnutrin polypeptide. In some embodiments, a PTD is covalently linked to the carboxyl terminus of an exogenous desnutrin polypeptide.

Exemplary protein transduction domains include but are not limited to a minimal undecapeptide protein transduction domain (corresponding to residues 47-57 of HIV-1 TAT comprising YGRKKRRQRRR; SEQ ID NO:19); a polyarginine sequence comprising a number of arginines sufficient to direct entry into a cell (e.g., 3, 4, 5, 6, 7, 8, 9, 10, or 10-50 arginines); a VP22 domain (Zender et al., Cancer Gene Ther. 2002 June; 9(6):489-96); an Drosophila Antennapedia protein transduction domain (Noguchi et al., Diabetes 2003; 52(7):1732-1737); a truncated human calcitonin peptide (Trehin et al. Pharm. Research, 21:1248-1256, 2004); polylysine (Wender et al., PNAS, Vol. 97:13003-13008); RRQRRTSKLMKR (SEQ ID NO:20); Transportan GWTLNSAGYLLGKINLKALAALAKKIL (SEQ ID NO:21); KALAWEAKLAKALAKALAKHLAKALAKALKCEA (SEQ ID NO:22); and RQIKIWFQNRRMKWKK (SEQ ID NO:23). Exemplary PTDs include but are not limited to, YGRKKRRQRRR (SEQ ID NO:19), RKKRRQRRR (SEQ ID NO:24); an arginine homopolymer of from 3 arginine residues to 50 arginine residues; Exemplary PTD domain amino acid sequences include, but are not limited to, any of the following: YGRKKRRQRRR (SEQ ID NO:19); RKKRRQRR (SEQ ID NO:25); YARAAARQARA (SEQ ID NO:26); THRLPRRRRRR (SEQ ID NO:27); and GGRRARRRRRR (SEQ ID NO:28).

The exogenous desnutrin polypeptide can be purified, e.g., at least about 75% pure, at least about 80% pure, at least about 85% pure, at least about 90% pure, at least about 95% pure, at least about 98% pure, at least about 99% pure, or more than 99% pure, e.g., free of proteins other than the desnutrin polypeptide being introduced into the cell and free of macromolecules other than the desnutrin polypeptide being introduced into the cell.

Introduction of an Exogenous Desnutrin Nucleic Acid into an Adipocyte

In some embodiments, a subject method involves introducing into an adipocyte an exogenous nucleic acid comprising a nucleotide sequence encoding a desnutrin polypeptide. Such an exogenous nucleic acid is also referred to herein as an “exogenous desnutrin nucleic acid.”

The exogenous nucleic acid comprising a nucleotide sequence encoding an exogenous denustrin polypeptide can be a recombinant expression vector, where suitable vectors include, e.g., recombinant retroviruses, lentiviruses, and adenoviruses; retroviral expression vectors, lentiviral expression vectors, nucleic acid expression vectors, and plasmid expression vectors. In some cases, the exogenous nucleic acid is integrated into the genome of an adipocyte and its progeny. In other cases, the exogenous nucleic acid persists in an episomal state in the host adipocyte and its progeny. In some cases, an endogenous, natural version of the denustrin polypeptide-encoding nucleic acid may already exist in the cell but an additional “exogenous gene” (exogenous desnutrin nucleic acid) is added to the host adipocyte to increase expression of the desnutrin polypeptide. In other cases, the exogenous desnutrin polypeptide-encoding nucleic acid encodes a denustrin polypeptide having an amino acid sequence that differs by one or more amino acids from a polypeptide encoded by an endogenous desnutrin polypeptide-encoding nucleic acid within the host adipocyte.

In some embodiments, a population of adipocytes is contacted with an exogenous desnutrin nucleic acid, thereby genetically modifying adipocytes in the population. Where a population of adipocytes is genetically modified (in vitro or in vivo) with an exogenous desnutrin nucleic acid, the exogenous desnutrin nucleic acid can be introduced into greater than 20% of the total population of adipocytes, e.g., 25%, 30%, 35%, 40%, 44%, 50%, 57%, 62%, 70%, 74%, 75%, 80%, 90%, or other percent of cells greater than 20%.

In some embodiments, exogenous desnutrin nucleic acid is an expression construct (a recombinant expression construct) that provides for production of the encoded desnutrin polypeptide in the genetically modified adipocyte. In some embodiments, the expression construct is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g. viral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641648, 1999; Ali et al., Hum Mol Genet 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a desnutrin-encoding nucleotide sequence is operably linked to a control element, e.g., a transcriptional control element, such as a promoter. The transcriptional control element is functional in a eukaryotic cell, e.g., a mammalian cell. Suitable transcriptional control elements include promoters and enhancers. In some embodiments, the promoter is constitutively active. In other embodiments, the promoter is inducible.

Non-limiting examples of suitable eukaryotic promoters (promoters functional in a eukaryotic cell) include cytomegalovirus (CMV) immediate early, herpes simplex virus (HSV) thymidine kinase, early and late SV40, long terminal repeats (LTRs) from retrovirus, and mouse metallothionein-I.

In some embodiments, a desnutrin-encoding nucleotide sequence is operably linked to an adipocyte-specific control element. Adipocyte-specific control elements can include, e.g., an aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Selection of the appropriate vector and promoter is well within the level of ordinary skill in the art. The expression vector may also contain a ribosome binding site for translation initiation and a transcription terminator. The expression vector may also include appropriate sequences for amplifying expression.

Examples of suitable mammalian expression vectors (expression vectors suitable for use in mammalian host cells) include, but are not limited to: recombinant viruses, nucleic acid vectors, such as plasmids, bacterial artificial chromosomes, yeast artificial chromosomes, human artificial chromosomes, cDNA, cRNA, and polymerase chain reaction (PCR) product expression cassettes.

Examples of suitable viral vectors include, but are not limited, viral vectors based on retroviruses (including lentiviruses); adenoviruses; and adeno-associated viruses. An example of a suitable retrovirus-based vector is a vector based on murine moloney leukemia virus (MMLV); however, other recombinant retroviruses may also be used, e.g., Avian Leukosis Virus, Bovine Leukemia Virus, Murine Leukemia Virus (MLV), Mink-Cell focus-Inducing Virus, Murine Sarcoma Virus, Reticuloendotheliosis virus, Gibbon Abe Leukemia Virus, Mason Pfizer Monkey Virus, or Rous Sarcoma Virus, see, e.g., U.S. Pat. No. 6,333,195.

In other cases, the retrovirus-based vector is a lentivirus-based vector, (e.g., Human Immunodeficiency Virus-1 (HIV-1); Simian Immunodeficiency Virus (SIV); or Feline Immunodeficiency Virus (FIV)), See, e.g., Johnston et al., (1999), Journal of Virology, 73(6):4991-5000 (FIV); Negre D et al., (2002), Current Topics in Microbiology and Immunology, 261:53-74 (SIV); Naldini et al., (1996), Science, 272:263-267 (HIV).

The recombinant retrovirus may comprise a viral polypeptide (e.g., retroviral env) to aid entry into the target cell. Such viral polypeptides are well established in the art, see, e.g., U.S. Pat. No. 5,449,614. The viral polypeptide may be an amphotropic viral polypeptide, e.g., amphotropic env, which aids entry into cells derived from multiple species, including cells outside of the original host species. The viral polypeptide may be a xenotropic viral polypeptide that aids entry into cells outside of the original host species. In some embodiments, the viral polypeptide is an ecotropic viral polypeptide, e.g., ecotropic env, which aids entry into cells of the original host species.

Examples of viral polypeptides capable of aiding entry of retroviruses into cells include but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env, RD114, FeLV-C, FeLV-B, MLV 10A1 env gene, and variants thereof, including chimeras. See e.g., Yee et al., (1994), Methods Cell Biol., Pt A:99-112 (VSV-G); U.S. Pat. No. 5,449,614. In some cases, the viral polypeptide is genetically modified to promote expression or enhanced binding to a receptor.

In general, a recombinant virus is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. The retroviral packaging cell may comprise a gene encoding a viral polypeptide, e.g., VSV-g that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as E1A or E1B or other adenoviral proteins. For example, proteins supplied by packaging cells may be retrovirus-derived proteins such as gag, pol, and env; lentivirus-derived proteins such as gag, pol, env, vpr, vpu, vpx, vif, tat, rev, and nef; and adenovirus-derived proteins such as E1A and E1B. In many examples, the packaging cells supply proteins derived from a virus that differs from the virus from which the viral vector derives.

Packaging cell lines include but are not limited to any easily-transfectable cell line. Packaging cell lines can be based on 293T cells, NIH3T3, COS or HeLa cell lines. Packaging cells are often used to package virus vector plasmids deficient in at least one gene encoding a protein required for virus packaging. Any cells that can supply a protein or polypeptide lacking from the proteins encoded by such virus vector plasmid may be used as packaging cells. Examples of packaging cell lines include but are not limited to: Platinum-E (Plat-E); Platinum-A (Plat-A); BOSC 23 (ATCC CRL 11554); and Bing (ATCC CRL 11270), see, e.g., Morita et al., (2000), Gene Therapy, 7:1063-1066; Onishi et al., (1996), Experimental Hematology, 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

The retroviral construct may be derived from a range of retroviruses, e.g., MMLV, HIV-1, SIV, FIV, or other retrovirus described herein. The retroviral construct may encode all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides can help facilitate viral entry. In an exemplary embodiment, the recombinant retrovirus is HIV-1 virus comprising a VSV-g polypeptide but not comprising a HIV-1 env polypeptide.

The retroviral construct may comprise: a promoter, a multi-cloning site, and/or a resistance gene. Examples of promoters include but are not limited to CMV, SV40, EF1α, β-actin; retroviral LTR promoters, and inducible promoters. The retroviral construct may also comprise a packaging signal (e.g., a packaging signal derived from the MFG vector; a psi packaging signal). Examples of some retroviral constructs known in the art include but are not limited to: pMX, pBabeX or derivatives thereof. See e.g., Onishi et al., (1996), Experimental Hematology, 24:324-329. In some cases, the retroviral construct is a self-inactivating lentiviral vector (SIN) vector, see, e.g., Miyoshi et al., (1998), J. Virol., 72(10):8150-8157. In some cases, the retroviral construct is LL-CG, LS-CG, CL-CG, CS-CG, CLG or MFG. Miyoshi et al., (1998), J. Virol., 72(10):8150-8157; Onishi et al., (1996), Experimental Hematology, 24:324-329; Riviere et al., (1995), PNAS, 92:6733-6737. Virus vector plasmids (or constructs), include: pMXs, pMxs-IB, pMXs-puro, pMXs-neo (pMXs-IB is a vector carrying the blasticidin-resistant gene in stead of the puromycin-resistant gene of pMXs-puro) Kimatura et al., (2003), Experimental Hematology, 31: 1007-1014; MFG Riviere et al., (1995), Proc. Natl. Acad. Sci. U.S.A., 92:6733-6737; pBabePuro; Morgenstern et al., (1990), Nucleic Acids Research, 18:3587-3596; LL-CG, CL-CG, CS-CG, CLG Miyoshi et al., (1998), Journal of Virology, 72:8150-8157 and the like as the retrovirus system, and pAdexl Kanegae et al., (1995), Nucleic Acids Research, 23:3816-3821 and the like as the adenovirus system. In exemplary embodiments, the retroviral construct comprises blasticidin (e.g., pMXs-IB), puromycin (e.g., pMXs-puro, pBabePuro); or neomycin (e.g., pMXs-neo). See, e.g., Morgenstern et al., (1990), Nucleic Acids Research, 18:3587-3596.

Methods of producing recombinant viruses from packaging cells and their uses are well established; see, e.g., U.S. Pat. Nos. 5,834,256; 6,910,434; 5,591,624; 5,817,491; 7,070,994; and 6,995,009. Many methods begin with the introduction of a viral construct into a packaging cell line. The viral construct may be introduced into a host fibroblast by any method known in the art, including but not limited to: a calcium phosphate method, a lipofection method (Feigner et al. (1987) Proc. Natl. Acad. Sci. U.S.A. 84:7413-7417), an electroporation method, microinjection, Fugene transfection, and the like, and any method described herein.

A nucleic acid construct can be introduced into a host cell (e.g., an adipocyte) using a variety of well known techniques, such as non-viral based transfection of the cell. In an exemplary aspect the construct is incorporated into a vector and introduced into a host cell. Introduction into the cell may be performed by any non-viral based transfection known in the art, such as, but not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include transfection reagents such as Lipofectamine™, Dojindo Hilymax™, Fugene™, jetPEI™, Effectene™, and DreamFect™

### Methods of Treating Obesity

As noted above, a subject method for increasing BAT or converting WAT to BAT in an individual is useful for treating obesity. Thus, the present disclosure provides methods of treating obesity in an individual, the methods generally involving administering to the individual an effective amount of a desnutrin polypeptide or a nucleic acid comprising a nucleotide sequence encoding a desnutrin polypeptide (“a desnutrin nucleic acid”), as described above.

In some embodiments, an “effective amount” of a desnutrin polypeptide or a desnutrin nucleic acid is an amount that, when administered in one or more doses, is effective to achieve one or more of: a) conversion of WAT into BAT; b) reduction of WAT; c) increase the BAT:WAT ratio.

Individuals who are suitable for treatment with a subject method include individuals having body mass index (BMI) greater than about 25 k g/m², greater than about 27 kg/m², greater than about 30 kg/m², or greater than about 35 kg/m².

### Formulations, Dosages, and Routes of Administration

As discussed above, a subject treatment method generally involves administering to an individual in need thereof an effective amount of a desnutrin polypeptide or a nucleic acid comprising a nucleotide sequence encoding a desnutrin polypeptide. Formulations, dosages, and routes of administration are discussed below. For the purposes of the discussion of formulations, dosages, and routes of administration, the term “active agent” refers to a desnutrin polypeptide or a nucleic acid comprising a nucleotide sequence encoding a desnutrin polypeptide. In some instances, a composition comprising an active agent can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins

Suitable formulations at least in part depend upon the use or the route of entry, for example, parenteral, oral, or transdermal. The term “parenteral” as used herein includes percutaneous, subcutaneous, intravascular (e.g., intravenous), intramuscular, intraperitoneal injection, administration via infusion, and the like.

In one embodiment, an active agent is administered to a subject by systemic administration in a pharmaceutically acceptable composition or formulation. By “systemic administration” is meant in vivo systemic absorption or accumulation of drugs in the blood stream to facilitate distribution through the body. Systemic administration routes include, e.g., intravenous, subcutaneous, portal vein, intraperitoneal, inhalation, oral, intrapulmonary and intramuscular.

Formulations of agents can also be administered orally, topically, parenterally, by inhalation or spray, or rectally in dosage unit formulations containing pharmaceutically acceptable carriers, adjuvants and/or vehicles. Pharmaceutically acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated herein by reference. For example, preservatives, stabilizers, dyes and flavoring agents can be provided. These include sodium benzoate, sorbic acid and esters of p-hydroxybenzoic acid. In addition, antioxidants and suspending agents can be used.

Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., 1990. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for an active agent include ethylene-vinyl acetate copolymer particles, osmotic pumps, implantable infusion systems, and liposomes.

An active agent can be formulated for local delivery, e.g., delivery into, at, or near adipose tissue. As such, an active agent can be delivered subcutaneously (e.g., into or near subcutaneous WAT), into the abdominal cavity, etc.

Formulations suitable for oral administration can be in the form of discrete units such as capsules, gelatin capsules, sachets, tablets, troches, or lozenges, each containing a predetermined amount of the active agent; in the form of a powder or granules; in the form of a solution or a suspension in an aqueous liquid or non-aqueous liquid; or in the form of an oil-in-water emulsion or a water-in-oil emulsion. The therapeutic can also be administered in the form of a bolus, electuary or paste. A tablet can be made by compressing or molding the active agent optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Molded tablets can be made by molding, in a suitable machine, a mixture of the powdered drug and suitable carrier moistened with an inert liquid diluent.

Oral compositions generally include an inert diluent or an edible carrier. For the purpose of oral therapeutic administration, the active agent can be incorporated with excipients. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition can be sterile and can be fluid. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

In some embodiments, as described above, an active agent is a nucleic acid comprising a nucleotide sequence encoding a desnutrin polypeptide. Exemplary formulations and methods for the delivery of nucleic acids are known in the art. For example, nucleic acids can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. U.S. 2002130430), biodegradable nanocapsules, and bioadhesive microspheres, or by proteinaceous vectors (O'Hare and Normand, International PCT Publication No. WO 00/53722). In another embodiment, a nucleic acid is formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalacto-samine (PEI-PEG-triGAL) derivatives. In one embodiment, a nucleic acid is formulated as described in U.S. Patent Application Publication No. 20030077829, incorporated by reference herein in its entirety.

In one embodiment, a nucleic acid active agent is complexed with membrane disruptive agents such as those described in U.S. Patent Publication No. 2001/0007666, incorporated by reference herein in its entirety. In another embodiment, the membrane disruptive agent or agents and the nucleic acid active agent are also complexed with a cationic lipid or helper lipid molecule, such as those lipids described in U.S. Pat. No. 6,235,310, incorporated by reference herein in its entirety. In one embodiment, a nucleic acid active agent is complexed with delivery systems as described in US 2003/077829, WO 00/03683 and WO 02/087541, each incorporated herein by reference.

Where the active agent is a desnutrin polypeptide, the polypeptide can be delivered using any of a variety of known formulations and routes of administration. For example, a desnutrin polypeptide can be adsorbed onto a microparticle (see, e.g., U.S. Pat. No. 7,501,134) where the microparticle includes polymer such as a poly(α-hydroxy acid), a polyhydroxy butyric acid, a polycaprolactone, a polyorthoester, a polyanhydride, or a polycyanoacrylate; a polypeptide can be formulated with a hydrogel; and the like. The microparticle or the hydrogel can be biodegradable. For example, the desnutrin polypeptide can be incorporated into a hydrogel, such as a poly(lactic-co-glycolic acid) (PLGA) hydrogel, a polyurethane hydrogel, a poly(ethyleneglycol) hydrogel, a dextran hydrogel, a hyaluronic acid hydrogel, and the like. For suitable microparticles and hydrogels, see, e.g., U.S. Pat. No. 7,744,866.

Pharmaceutical compositions can be formulated for controlled or sustained delivery in a manner that provides local concentration of an active agent (e.g., bolus, depot effect) and/or increased stability or half-life in a particular local environment. The compositions can include the formulation of desnutrin polypeptides or desnutrin nucleic acids with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping an active agent, where the active agent is a desnutrin polypeptide.

Oral administration can be accomplished using pharmaceutical compositions containing an active agent (e.g., such as a desnutrin polypeptide, a nucleic acid comprising a nucleotide sequence encoding a desnutrin polypeptide) formulated as tablets, lozenges, aqueous or oily suspensions, dispersible powders or granules, emulsion, hard or soft capsules, or syrups or elixirs. Such oral compositions can contain one or more such sweetening agents, flavoring agents, coloring agents or preservative agents in order to provide pharmaceutically elegant and palatable preparations. Tablets, which can be coated or uncoated, can be formulated to contain the active ingredient in admixture with non-toxic pharmaceutically acceptable excipients, e.g., inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. Where a coating is used, the coating can delay disintegration and absorption in the gastrointestinal tract and thereby provide a sustained action over a longer period.

Where the formulation is an aqueous suspension, such can contain the active agent in a mixture with a suitable excipient(s). Such excipients can be, as appropriate, suspending agents (e.g., sodium carboxymethylcellulose, methylcellulose, hydropropyl-methylcellulose, sodium alginate, polyvinylpyrrolidone, gum tragacanth and gum acacia); dispersing or wetting agents; preservatives; coloring agents; and/or flavoring agents.

Dosage levels can be readily determined by the ordinarily skilled clinician, and can be modified as required, e.g., as required to achieve the desired effect. Dosage levels can be on the order of from about 0.1 mg to about 100 mg per kilogram of body weight per day. The amount of active agent that can be combined with the carrier materials to produce a single dosage form varies depending upon, e.g., the host treated and the particular mode of administration. Dosage unit forms can contain between from about 1 mg to about 500 mg of an active agent.

An active agent can be delivered via any of a variety of modes and routes of administration, including, e.g., local delivery by injection; local delivery by continuous release; systemic delivery by oral administration; systemic delivery by intravenous administration; and the like. An active agent can be delivered intraperitoneally.

### Screening Methods

The present disclosure provides a method of identifying an agent that increases desnutrin levels and/or activity. An agent thus identified is a candidate agent for increasing the BAT:WAT ratio in an individual. As such, the present disclosure provides methods of identifying candidate agents for increasing the BAT:WAT ratio in an individual. A test agent that increases the level and/or activity of desnutrin is considered a candidate agent for converting WAT to BAT. A test agent that increases the level and/or activity of desnutrin is considered a candidate agent for treating obesity.

In some cases, the methods involve contacting a PNPLA2 (desnutrin) polypeptide with a test agent in vitro; and determining the effect, if any, of the test agent on PNPLA2 levels and/or activity. A test agent that increases PLPLA2 levels and/or activity is considered a candidate agent for increasing BAT:WAT ratio in an individual. Increasing the BAT:WAT ratio in an individual can be used to treat obesity.

A subject screening method can be carried out as a cell-free in vitro assay, e.g., using a PNPLA2 polypeptide. A subject screening method can also be carried out as a cell-based in vitro assay, e.g., using a cell that produces PNPLA2.

A subject screening method generally includes appropriate controls, e.g., a control sample that lacks the test agent. Generally a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents such as salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

As used herein, the term “determining” refers to both quantitative and qualitative determinations and as such, the term “determining” is used interchangeably herein with “assaying,” “measuring,” and the like.

The terms “candidate agent,” “test agent,” “agent”, “substance” and “compound” are used interchangeably herein. Candidate agents encompass numerous chemical classes, including synthetic, semi-synthetic, and naturally occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.) and can also be used. Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 daltons and less than about 2,500 daltons. Candidate agents may comprise functional groups necessary for structural interaction with proteins, e.g., hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

A test agent can be a small molecule. The test molecules may be individual small molecules of choice or in some cases, the small molecule test agents to be screened come from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See, e.g., Gallop et al., (1994), J. Med. Chem., 37(9), 1233-1251. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al., (1993), Proc. Natl. Acad. Sci. U.S.A., 90:6909-6913; analogous organic syntheses of small compound libraries, as described in Chen et al., (1994), J. Amer. Chem. Soc., 116:2661-2662; Oligocarbamates, as described in Cho, et al., (1993), Science, 261:1303-1305; peptidyl phosphonates, as described in Campbell et al., (1994), J. Org. Chem., 59: 658-660; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

### Cell-Free In Vitro Assay

As noted above, in some embodiments, a subject screening method is a cell-free in vitro assay. The methods generally involve contacting a desnutrin polypeptide in vitro with a test agent and with a substrate for desnutrin; and determining the effect, if any, of the test agent on the enzymatic activity of the desnutrin polypeptide.

A test agent of interest is one that increases desnutrin enzymatic activity by at least about 20%, at least about 25%, at least about 30%, at least about 40%, at least about 50%, at least about 60%, at least about 70%, at least about 80%, at least about 90%, at least about 2-fold, at least about 2.5-fold, at least about 3-fold, at least about 5-fold, at least about 10-fold, or more than 10-fold, compared to the enzymatic activity of the desnutrin polypeptide in the absence of the test agent. In some embodiments, the desnutrin polypeptide is substantially pure, e.g., at least about 75%, at least about 80%, at least about 85%, at least about 90%, at least about 95%, at least about 98%, or greater than 98%, pure.

In some embodiments, a test agent of interest is one that increases desnutrin enzymatic activity with a half-maximal effective concentration (EC₅₀) of from about 100 μM to about 50 μM, from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

A subject method generally involves contacting a test agent with a desnutrin polypeptide and a substrate for desnutrin. Enzymatic activity is assessed by detecting the product of desnutrin activity on the desnutrin substrate. Suitable substrates include any triacylglycerol. Detection of a diacylglycerol and/or a free fatty acid product of the desnutrin activity on the TAG provides an indication of the effect of the test agent on desnutrin enzymatic activity. One or more of the fatty acids in the TAG can include a radioactive label, to provide for detection of the fatty acid upon release from the TAG substrate.

Assays for desnutrin enzymatic activity are known in the art. See, e.g., Duncan, R. E., Wang, Y., Ahmadian, M., Lu, J., Sarkadi-Nagy, Sul, H S. J Lipid Res 2010, 51, 309-17, Characterization of Desnutrin Functional Domains: Critical Residues for Triacylglycerol Hydrolysis in Cultured Cells. As one non-limiting example, lysates are prepared from cells or tissue by lysis in 50 mM Tris, pH 7.4, 0.1 M sucrose, and 1 mM ethylenediaminetetraacetic acid (EDTA), followed by centrifugation at 16,000×g for 15 minutes at 4° C. Reactions are started by addition of supernatants containing 50-100 μg of protein in 100 μl volumes to 100 μl of 2× concentrations of triolein substrate containing [³H]triolem as radioactive tracer, sonicated into mixed micelles with 25 μM egg yolk lecithin, 100 μM taurocholate, 2% bovine serum albumin (BSA) (w/v), 2 mM EDTA, 1 mM dithiothreitol (DTT), and 50 mM potassium phosphate, pH 7.2. Reactions are allowed to proceed for 15-60 minutes at 37° C. and are terminated by the addition of 1.25 ml of methanol:chloroform:heptane (10:9:7). Fatty acids are extracted with 0.5 ml of 0.1 M potassium carbonate, 0.1 M boric acid, pH 10.5, and radioactivity in the upper phase obtained after centrifugation for 20 min at 800×g is quantified by liquid scintillation counting.

A test agent of interest is assessed for any cytotoxic activity (other than anti-proliferative activity) it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity are considered candidate agents.

A test agent that increases PNPLA2 levels and/or activity can be subjected to further assays, e.g., in vivo assays. For example, a test agent that increases PNPLA2 levels and/or activity can be administered to an experimental animal model; and the effect, if any, of the agent on the BAT:WAT ratio can be assessed.

### Cell-Based Assay

In some embodiments, a subject screening method is an in vitro cell-based assay for identifying an agent that increases the activity and/or level of desnutrin in a cell. The method generally involves contacting a cell that produces desnutrin with a test agent; and determining the effect, if any, of the test agent on the level and/or activity of desnutrin in the cell. The assay can further involve determining the level of a BAT-selective gene product in the cell. BAT-selective gene products, and methods for detecting same, are described above.

In some embodiments, the cells (“host cells”) used in the assays are mammalian cells. Suitable host cells include eukaryotic host cells that can be cultured in vitro, either in suspension or as adherent cells.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

The cell used in the assay can produce desnutrin endogenously. The cell used in the assay can be genetically modified with a recombinant expression vector comprising a nucleotide sequence encoding desnutrin, such that the encoded desnutrin is produced in the cell. In general, the genetically modified cells can be produced using standard methods. Expression constructs comprising nucleotide sequences encoding a desnutrin polypeptide are introduced into the host cell using standard methods practiced by one with skill in the art. In some embodiments, the desnutrin polypeptide is encoded on a transient expression vector (e.g., the vector is maintained in an episomal manner by the cell). Alternatively, or in addition, a desnutrin polypeptide-encoding expression construct can be stably integrated into the cell line.

The effect of the test agent on the level of desnutrin in the cell can be determined using any of a variety of assays. For example, an immunological assay (e.g., an ELISA, an RIA, etc.) can be used to determine the level of desnutrin polypeptide in the cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Ablation of Desnutrin/ATGL in Adipose Tissue Promotes Obesity and a Brown-to-White Adipose Phenotype

### Experimental Procedures

### Mouse Maintenance

All studies received approval from the University of California at Berkeley Animal Care and Use Committee. Desnutrin-ASKO and flox/flox littermates on a C57BL/6J background were compared. Either a standard chow or a high fat diet (HFD) (45% of kcal from fat, 35% of kcal from carbohydrate and 20% of kcal from protein, Research Dyets) was provided ad libitum. All studies, unless indicated otherwise, were performed on high-fat diet fed mice.

### Indirect Calorimetry and Body Temperature

Oxygen consumption (VO2) was measured using the Comprehensive Laboratory Animal Monitoring System (CLAMS; Columbus Instruments). Data were normalized to body weights. Body temperatures were assessed in 25 wk-old male mice using a RET-3 rectal probe for mice (Physitemp). CL31624 was intraperitoneally injected into mice at 1 mg/kg body weight.

### Glucose and Insulin Tolerance Tests

For the glucose tolerance test (GTT), mice were injected intraperitoneally with D-glucose (2 mg/g body weight) after an overnight fast and monitored tail blood glucose levels. For insulin tolerance test (ITT), mice were intraperitoneally injected with insulin (humulin, Eli Lilly) (0.75 mU per g body weight) after a 5-h fast.

### Adipocyte Size Determination

Gonadal fat samples and intrascapular BAT were fixed in 10% buffered formalin, embedded in paraffin, cut into 8 μm-thick sections, and stained with hemotoxylin and eosin. Adipocyte size was determined with Image J software (US National Institutes of Health), measuring a minimum of 300 cells per sample.

### Lipolysis

Gonadal fat pads or BAT from overnight fasted mice were cut into 50 mg samples and incubated at 37° C. without shaking in 5000 of Krebs-Ringer buffer (12 mM HEPES, 121 mM NaCl, 4.9 mM KCl, 1.2 mM MgSO₄ and 0.33 mM CaCl₂) containing 2% fatty acid free bovine serum albumin (BSA) and 0.1% glucose with or without 10 μM isoproterenol. Fatty acid (FA) and glycerol release were measured in aliquots from incubation buffer using the NEFA C Kit (Wako) and Free Glycerol Reagent (Sigma), respectively. For reconstitution of lipolysis in transfected HEK 293-FT cells, 293-FT cells were plated in 6-well plates and transfected with either green fluorescent protein (GFP), wild type desnutrin-HA-GFP or mutant desnutrin 5406A-HA-GFP. Four hours later the transfection mixture was removed and the cells were treated with growth medium containing 300 μM oleic acid, 1% BSA, 0.5 μg/ml insulin for 16 hrs. The cells were rinsed once with Krebs-Ringer buffer supplemented with 4% fatty acid-free BSA and then incubated in this media overnight. Glycerol and fatty acid were determined using the kits described above.

### RNA Extraction and Real Time RT-PCR

Total RNA was prepared using Trizol Reagent (Invitrogen) and cDNA was synthesized from 2.5 μg of total RNA by Superscript II reverse transcriptase (Invitrogen). Gene expression was determined by reverse transcription-quantitative polymerase chain reaction (RT-qPCR) performed with an ABI PRISM7700 sequence fast detection system (Applied Biosystems), and was quantified by measuring the threshold cycle normalized to glyceraldehyde-3-phosphate dehydrogenase (GAPDH) then expressed relative to flox/flox controls.

### Immunoblotting

Total lysates were subjected to 8% sodium dodecyl sulfate-polyacrylamide gel electrophoresis (SDS-PAGE), transferred to nitrocellulose membranes, and probed with rabbit anti-desnutrin antibody that was generated, anti-GAPDH antibody (Santa Cruz), UCP-1 antibody (Sigma) followed by horse-radish peroxidase conjugated secondary antibody (Biorad). Blots were visualized using enhanced chemiluminescence substrate (PerkinElmer) and images were captured using a Kodak Image Station 4000MM.

### Transmission Electron Microscopy

BAT and WAT were fixed in 2% glutaraldehyde in 0.1 M PB (phosphate buffer), pH 7.3 at 4° C. overnight; then postfixed in 1% OsO₄ and embedded in an Epon-Araldite mixture. Ultrathin sections (0.2 μm) mounted on 150-mesh copper grids were stained with lead citrate and observed under a FEI Tecnai 12 transmission electron microscope.

### Indirect Calorimetry and Body Temperature

Oxygen consumption (VO2) was measured using the Comprehensive Laboratory Animal Monitoring System (CLAMS: Columbus Instruments). Data were normalized to body weights. Body temperatures were assessed using a RET-3 rectal probe for mice (Physitemp).

### Blood and Tissue Metabolites-Serum Parameters

Fasting serum triglycerides and FAs were analyzed with Infinity Triglyceride reagent (Thermo Trace) and NEFA C kit (Wako), respectively. Serum insulin, were determined using enzyme-linked immunosorbent assay kits (Alpco).

### ²H₂O Labeling and GCMS Analysis of TAG-Glycerol and TAG-FA

The heavy water (²H₂O) labeling protocol and gas chromatograph-mass spectrometry (GCMS) analyses of triacylglycerol (TAG)-glycerol and TAG-FA from adipose tissue have been described previously in detail (Turner et al., 2003). Mice were intraperitoneally injected with 100% ²H₂O, 0.9% NaCl (0.025 ml/g body weight) and administered ²H₂O in drinking water starting at 20 weeks of age for a 6 day period after which lipids were extracted from gonadal fat pads by the Folch method (Folch et al., 1957) for subsequent analysis.

Calculation of all-Source TAG Turnover

Fractional TAG-glycerol synthesized from glycerol phosphate during the period of ²H₂O administration was measured as described (Turner et al., 2003):

fTAG=EM1_{TAG-glycerol}/A1_{TAG-glycerol}.

EM1 is the measured excess mass isotopomer abundance for M1-glycerol at time t and A1 is the asymptotic mass isotopomer abundance for M1-glycerol, assuming that four of five C—H bonds of glycerol phosphate are replaced by H-atoms from tissue water (Turner et al., 2003).

### Calculation of De Novo Palmitate Turnover

Fractional contributions from de novo lipogenesis (DNL) were calculated using a combinatorial model as previously described (Turner et al., 2003):

fDNL=EM1_{FA}/A1_{FA}

where fDNL represents the fraction of total TAG-palmitate in the depot derived from DNL during the labeling period. The fraction of newly synthesized TAG-palmitate from DNL is also calculated by correcting the measured fractional contribution from DNL (fDNL) for the degree of replacement of adipose TAG during the labeling period:

DNL contribution to newly synthesized TAG=fDNL/fTAG.

### In Vitro Kinase Assay

HEK 293 cells were transfected with either GFP, wild type desnutrin-HA-GFP or mutant forms of desnutrin S406A-HA-GFP and S430A-HA-GFP, immunoprecipitated with anti-hemagglutinin (HA) antibody conjugated beads (Covance) and then incubated with purified AMPKα1(Millipore) carried out in a buffer containing 5 mM HEPES, pH 7.5, 0.1 mM dithiothreitol, 0.25% NonidetP-40, 7.5 mM MgCl₂, 50 μM ATP, 5 μCi of [γ-³²P]ATP and incubated for 30 min at 30° C. Reactions were stopped with the addition of 2×SDS loading buffer. The protein products were separated on SDS-PAGE, transferred to nitrocellulose membranes, which were then subjected to autoradiography and western blot analysis using an anti-HA antibody (Covance) or an anti-phospho-(Ser) 14-3-3 binding motif antibody (Cell Signaling).

### Hyperinsulinemic-Euglycemic Clamp

Jugular venous catheters were implanted seven days prior to the study. After an overnight fast, [3-³H]glucose (Perkin Elmer) was infused at a rate of 0.05 μCi/min for 2 hours to assess basal glucose turnover, followed by the hyperinsulinemic-euglycemic clamp for 140 min with a primed/continuous infusion of human insulin (154 pmol/kg prime (21 mU/kg)) over 3 min, followed by 17 pmol/kg/min (3 mU/kg/min) infusion (Novo Nordisk, Princeton, N.J.), a continuous infusion of [3-³H]glucose (0.1 μCi/min), and a variable infusion of 20% dextrose to maintain euglycemia (100-120 mg/dl). Plasma samples were obtained from the tail and measured tissue-specific glucose uptake after injection of a bolus of 10 μCi of 2-deoxy-D-[1-¹⁴C]glucose (Perkin Elmer) at 85 min. The results were analyzed as previously described (Samuel et al., 2006).

### Chromatin Immunoprecipitation

BAT was isolated as previously described and fixed with 2 mM DSG for 45 min at room temperature (RT) before 2% formaldehyde crosslinking for 30 min. Chromatin immunoprecipitation (ChIP) was performed as previously described (Latasa et al., 2003; Wong et al., 2009) using antibodies to GAPDH, PPARα and RIP140 (Santa Cruz) and primers to the UCP-1 enhancer (forward primer: AGCTTGCTGTCACTCCTCTACA (SEQ ID NO:29); reverse primer: TGAGGAAAGGGTTGACCTTG (SEQ ID NO:30)).

### Statistical Analyses

The results are expressed as means+/−SEM. Statistically significant differences between two groups were assessed by Student's t test.

### Results

### Adipose-Specific Ablation of Desnutrin Promotes Diet-Induced Obesity Due to Impaired Lipolysis and Thermogenesis

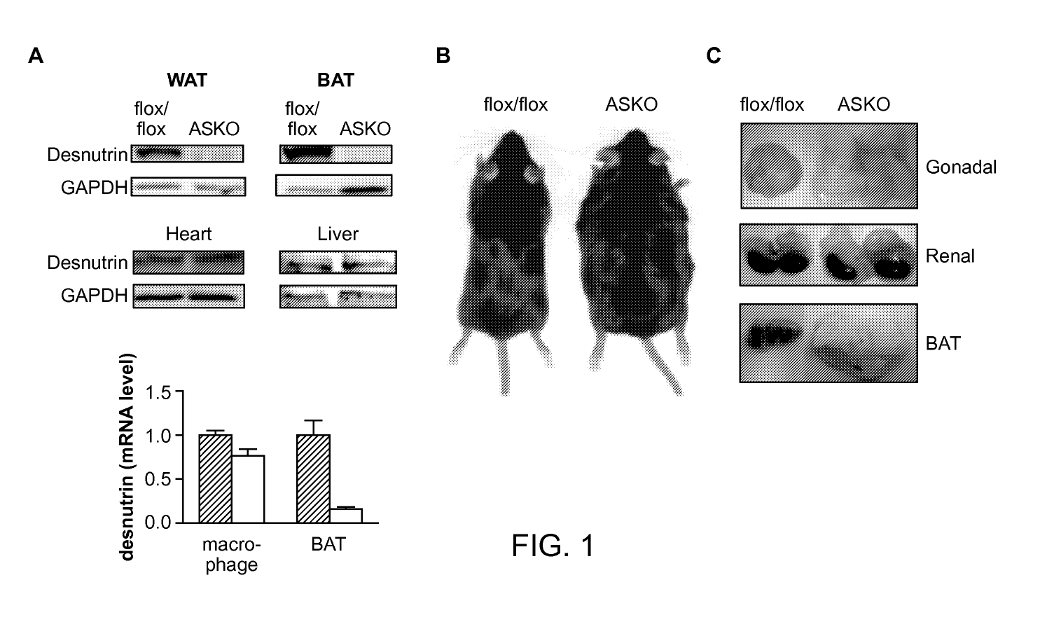

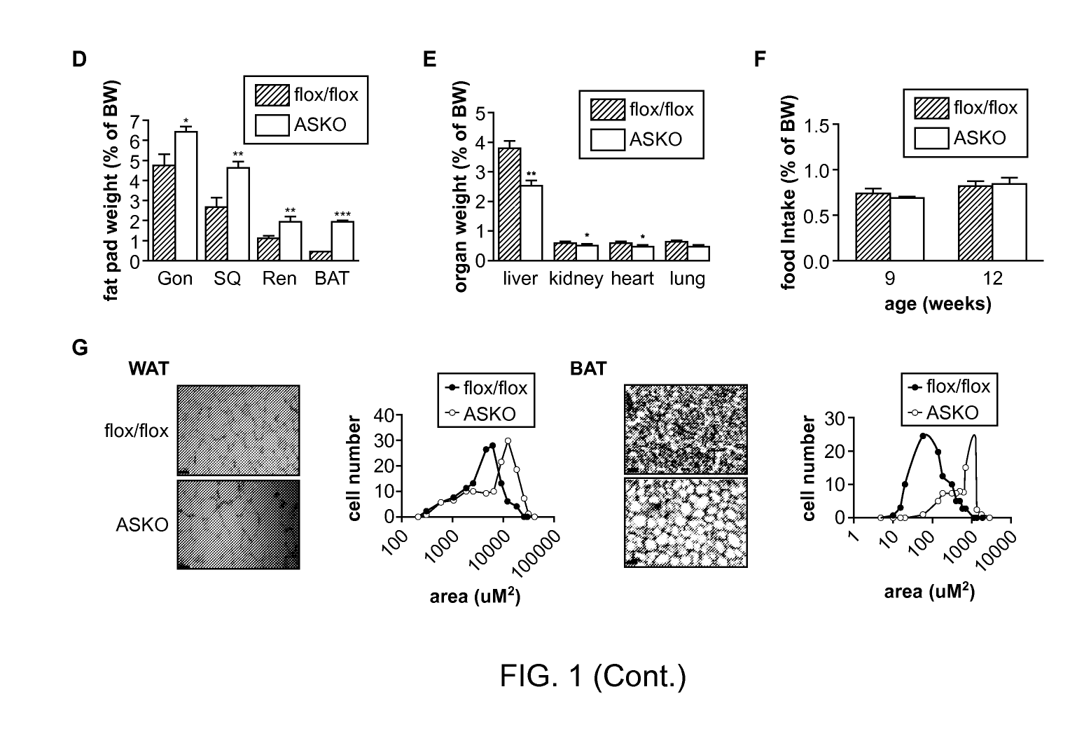

To determine the role of desnutrin and the physiological consequence of lack of desnutrin, specifically in adipose tissue, gene targeting was used to generate floxed mice that have the first exon of desnutrin containing the translational start site as well as the conserved lipase consensus motif (GXSXG) flanked by lox P sites (flox/flox mice). Flox/flox mice were subsequently crossed with aP2-Cre mice to generate desnutrin adipose-specific knockout (desnutrin-ASKO mice) and compared to flox/flox littermates for all experiments. Desnutrin-ASKO mice were born at the expected Mendelian frequency and exhibit a normal life expectancy. Using an antibody raised against desnutrin, western blot analysis verified that the desnutrin protein was not detected in WAT and BAT of desnutrin-ASKO mice but, as expected, was present in flox/flox control mice (FIG. 1A, upper). However, in other organs, such as the heart and liver, desnutrin protein levels were unchanged compared to flox/flox mice (FIG. 1A, middle). By RT-qPCR, minimal reduction in desnutrin in the macrophage, compared to BAT, was detected (FIG. 1A, lower).

Mice were given a high fat or standard chow diet at weaning. Although total body weights did not differ at weaning, by 11 weeks of age, desnutrin-ASKO mice fed a HFD began to gain weight at a higher rate than flox/flox littermates. Increased weight gain and fat pad weight was also observed in chow-fed desnutrin-ASKO mice, albeit to a lesser extent. However, there was no difference in food intake (FIG. 1F). Compared to flox/flox mice, weights of other organs such as liver, kidney and heart were not changed in desnutrin-ASKO mice fed a high fat diet and, therefore, could not account for the increased body weights in desnutrin-ASKO mice (FIG. 1E). However, WAT and BAT depot sizes were markedly enlarged in desnutrin-ASKO mice (FIGS. 1C and D). Gonadal, subcutaneous and renal WAT depot weights were 1.4, 1.7 and 1.9-fold higher, respectively, after 20 weeks on a HFD in desnutrin-ASKO mice compared to flox/flox mice (FIG. 1D). BAT was even more affected than WAT, weighing 5.3-fold more than flox/flox mice, and resembling WAT in terms of its pale color (FIGS. 1C and D). Histological analysis revealed a greater frequency of larger adipocytes in gonadal fat pads from desnutrin-ASKO mice indicating increased adipocyte size (FIG. 1G, left). Similarly, brown adipocyte size was also markedly increased in desnutrin-ASKO mice (FIG. 1G, right). Taken together, these findings indicate that desnutrin-ASKO mice exhibit increased adiposity with larger adipocyte size in both WAT and BAT.

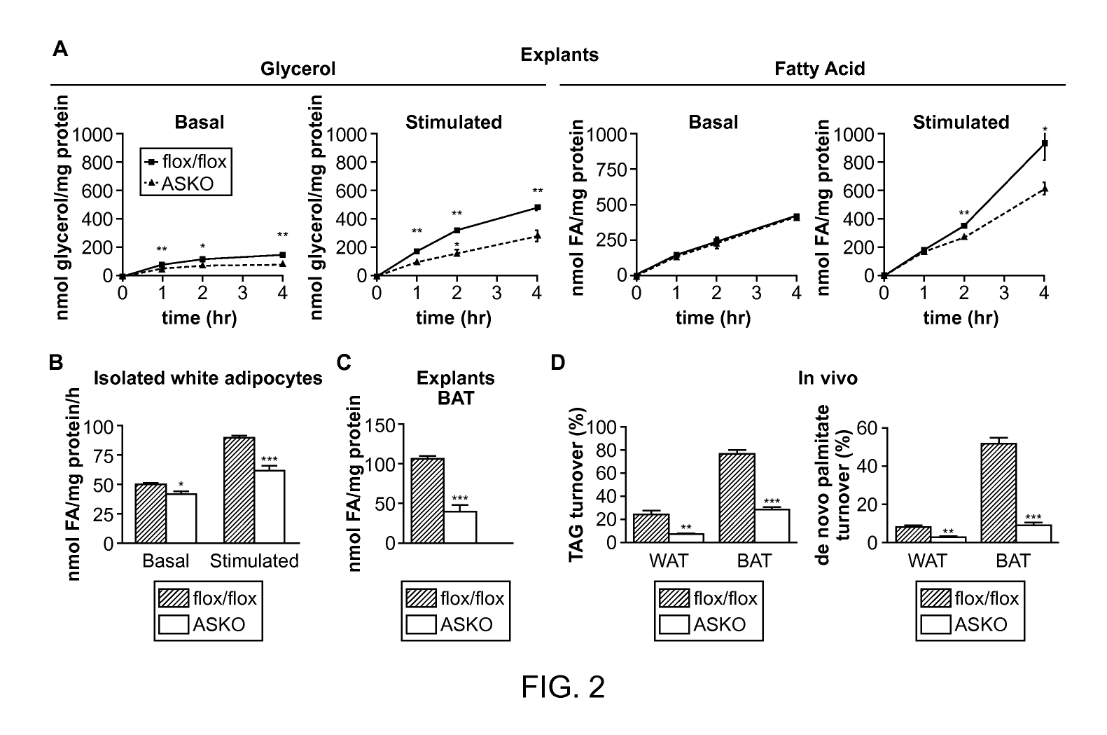

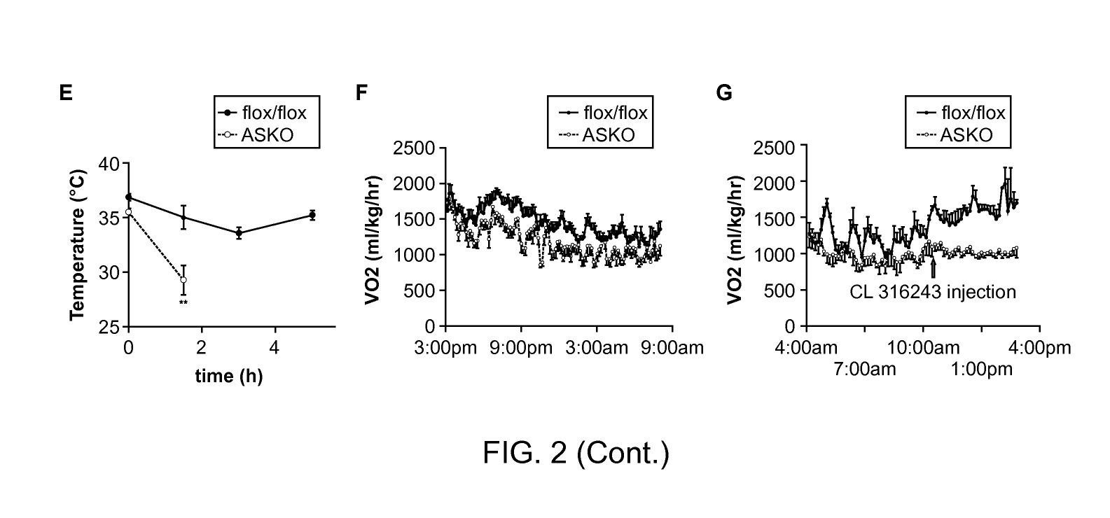

Given desnutrin is the major TAG hydrolase in adipose tissue, it was predicted that the increased adiposity observed in desnutrin-ASKO mice was due to impaired lipolysis. The expression levels of early as well as late markers of adipocyte differentiation were not changed in desnutrin-ASKO WAT, indicating normal adipocyte differentiation. Glycerol and FA release from explants of WAT of desnutrin-ASKO mice and flox/flox littermates were measured. Indeed, glycerol release followed over 4 hours was drastically decreased in desnutrin-ASKO WAT compared to flox/flox WAT under both basal and isoproterenol-stimulated conditions (FIG. 2A, left). Although FA release was not changed under basal conditions in WAT, it was decreased by 22% after 2 hours and 41% after 4 hours in desnutrin-ASKO WAT under isoproterenol-stimulated conditions (FIG. 2A, right). Furthermore, in isolated adipocytes, FA release was decreased under both basal and stimulated conditions (FIG. 2B). Lipolysis was also severely blunted in BAT of desnutrin-ASKO mice, being decreased by 60% under basal conditions (FIG. 2C). Using a recently developed heavy water labeling technique, in vivo TAG turnover and de novo palmitate turnover were measured over a 6-day period in WAT and BAT from flox/flox and desnutrin-ASKO mice. While TAG turnover was 24% after 6 days in WAT of flox/flox mice, it was 7% in desnutrin-ASKO mice (FIG. 2D, left). In flox/flox BAT, TAG turnover was much higher than in WAT, with 77% turnover after 6 days (FIG. 2D, left). However, in desnutrin-ASKO BAT, it was only 29%, which is similar to levels in WAT of flox/flox mice (FIG. 2D, left). Consistent with these findings, de novo palmitate turnover was 8% and 52% in WAT and BAT of flox/flox mice, respectively, compared to 3% and 9% in desnutrin-ASKO mice (FIG. 2D, right). Taken together, these findings indicate that lipolysis is severely impaired in both WAT and BAT of desnutrin-ASKO mice and other lipases in adipose tissue cannot compensate for lack of desnutrin.

Since lipolysis and FAs are critical for thermogenesis, it was predicted that the severely blunted lipolysis in desnutrin-ASKO mice, would lead to impaired thermogenesis. Desnutrin-ASKO mice and flox/flox littermates were subjected to cold stress. While flox/flox mice were able to maintain body temperature well into 5 hours at 4° C., desnutrin-ASKO mice quickly reached life-threatening hypothermia after just 90 min (FIG. 2E). While there was no change in activity levels between desnutrin-ASKO and flox/flox mice, total oxygen consumption was decreased in desnutrin-ASKO mice when mice were housed in metabolic chambers overnight in the fasted state (FIG. 2F). Since desnutrin-ASKO mice have impaired lipolysis and thermogenesis, it was hypothesized that administration of a β3 agonist, which signals through β3 adrenergic receptors during cold exposure to increase energy expenditure through stimulation of lipolysis, should no longer exert its thermogenic effects in desnutrin-ASKO mice (Cannon and Nedergaard, 2004). To test this, a β3-agonist, CL31624, was injected into desnutrin-ASKO and flox/flox mice and oxygen consumption was monitored. In response to CL31624 injection, flox/flox mice exhibited a drastic increase in their metabolic rate, as indicated by oxygen consumption, however, desnutrin-ASKO mice showed no change in oxygen consumption, revealing a blunted β3 adrenergic response (FIG. 2F). Taken together, these results indicate that BAT in desnutrin-ASKO mice is unresponsive to both physiological and pharmacological thermogenic stimulation, revealing the requirement of desnutrin for eliciting a proper β3 thermogenic response.

### Desnutrin Ablation Promotes the Conversion of BAT to WAT

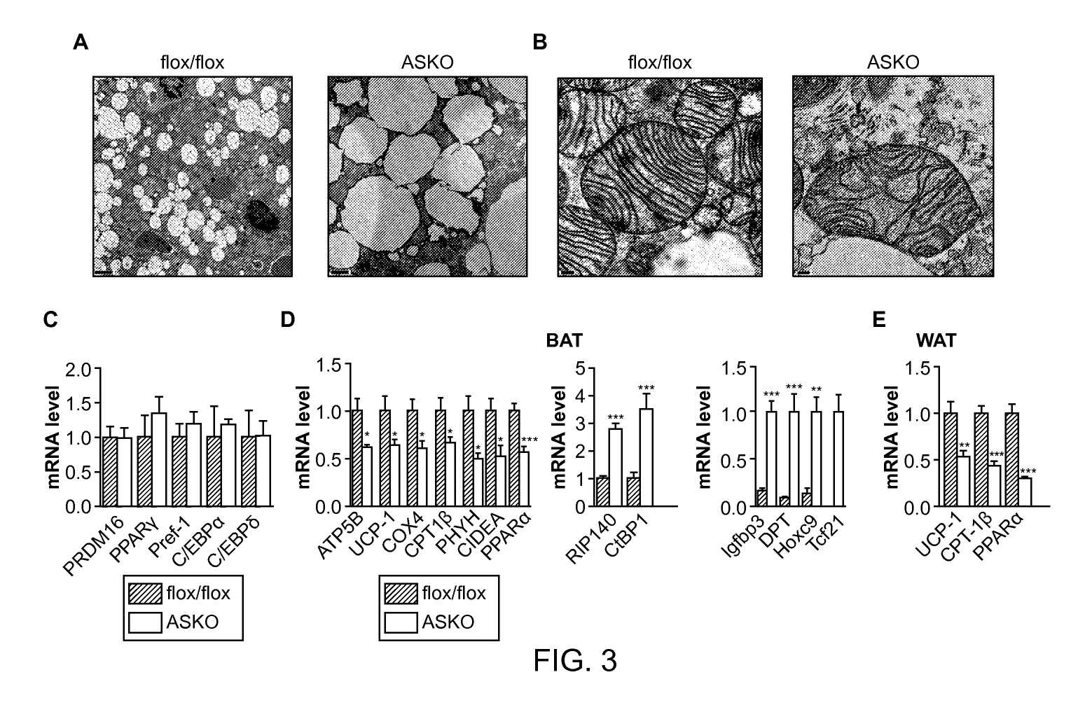

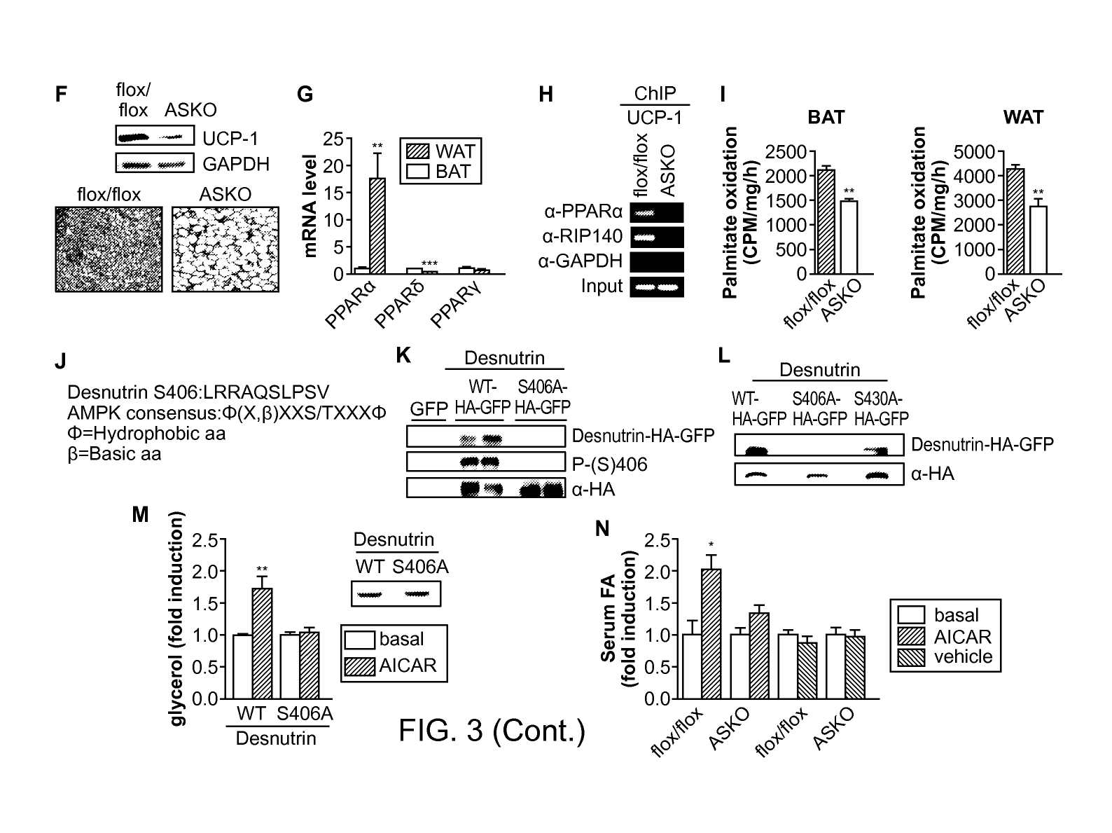

Impaired lipolysis in desnutrin-ASKO mice led to strikingly massive TAG accumulation in BAT and impaired thermogenesis. Using transmission electron microscopy a drastic difference was observed in the morphology of BAT between adult flox/flox and desnutrin-ASKO mice. While BAT from flox/flox mice had numerous small lipid droplets, BAT from desnutrin-ASKO mice contained larger, but fewer lipid droplets (FIG. 3A). Fewer mitochondria were also observed in BAT from desnutrin-ASKO mice and the majority of mitochondria were composed of randomly oriented cristae, characteristic of WAT, compared to the classic laminar cristae found in flox/flox BAT (FIG. 3B). However, BAT morphology was not altered in desnutrin-ASKO mice during embryogenesis at either E17 or E21, suggesting that the conversion of BAT to a WAT-like phenotype is likely due to the metabolic consequence of decreased lipolysis rather than a developmental defect. In this regard, BAT from desnutrin-ASKO mice showed no changes in the expression of Pref-1, C/EBPα, C/EBPδ, PPARγ as well as PRDM16, which has been shown to be important for brown adipocyte differentiation (FIG. 3C) (Seale et al., 2008). The expression of genes involved in thermogenesis, mitochondrial and peroxisomal FA oxidation was decreased compared to flox/flox mice. ATP5B, COXIV, CPT1β, PhyH, Cidea and PPARa were all decreased by 35-50% (FIG. 3D, left). Furthermore, UCP-1 expression was markedly decreased at both the mRNA and protein level, as shown by western blotting and immunostaining. (FIGS. 3D, left and 3F). RIP140 and CtBP1, transcriptional co-repressors that may play a role in suppressing oxidative and thermogenic genes in adipose tissue were upregulated by 2.8 and 3.5-fold, respectively in BAT of desnutrin-ASKO mice (FIG. 3D, middle) (Christian et al., 2005; Fruhbeck et al., 2009; Leonardsson et al., 2004). Furthermore, expression of WAT-enriched genes such as Igfbp3, DPT, Hoxc9 and Tcf21 were strongly induced in BAT of desnutrin-ASKO mice (FIG. 3D, right) (Petrovic et al.). Consistent with the findings in BAT, it was found that UCP-1, CPT1β and PPARα expression were also decreased in WAT of desnutrin-ASKO mice (FIG. 3E).

It is conceivable that lower FA levels within adipocytes due to blunted lipolysis in desnutrin-ASKO mice may affect the activity of PPARs that are known to be FA sensors in cells and control the expression of many oxidative and thermogenic genes (Evans et al., 2004). In this regard, by RT-qPCR it was found that, among the three PPAR members, only PPARα is expressed at a much higher level in BAT compared to WAT (FIG. 3G), and PPARα has been shown to activate the UCP-1 promoter (Barbera et al., 2001). In addition, ligand availability may influence PPARa binding to target promoters (Mandard et al., 2004; van der Meer et al.). Chromatin immunoprecipitation (ChIP) was performed with an anti-PPARα antibody in BAT of desnutrin-ASKO and flox/flox mice. Less PPARα was bound to the-2.5 kb enhancer region of the UCP-1 promoter in desnutrin-ASKO mice compared to flox/flox mice (FIG. 3H). Less RIP140 binding to the UCP-1 promoter was observed in desnutrin-ASKO BAT, despite the significantly higher expression levels. Although RIP140 has been reported to play a role in suppressing a BAT phenotype, it was predicted that impaired binding of PPARα may have precluded binding of this corepressor in our desnutrin-ASKO mice. It was previously found that increasing lipolysis promotes FA oxidation within adipocytes (Ahmadian et al., 2009b; Jaworski et al., 2009). Furthermore, decreased expression of oxidative genes in both WAT and BAT of desnutrin-ASKO mice was also observed. FA oxidation in isolated white and brown adipocytes from flox/flox and desnutrin-ASKO mice was compared by measuring the production of ¹⁴CO₂ from [¹⁴C]palmitate. Indeed, FA oxidation was blunted in both white and brown adipocytes from desnutrin-ASKO mice (FIG. 3I). Taken together, by suppressing lipolysis, ablation of desnutrin decreased FA oxidation within adipocytes and suppressed expression of UCP-1, with impaired PPARa binding to its promoter. As a result, a drastic change of BAT to a WAT-like phenotype, and impaired thermogenesis, were observed.

### Desnutrin is Phosphorylated by AMPK to Increase Lipolysis

It was found that by stimulating lipolysis, desnutrin promotes FA oxidation and thermogenesis in adipose tissue. However, how desnutrin activity is increased during a low energy state is unclear. AMPK, a master cellular energy sensor, is activated during a low energy state and has a well-established role in increasing FA oxidation through phosphorylation of ACC (Lage et al., 2008). However, its function in adipose tissue metabolism and in regulating lipolysis has been unclear (Koh et al., 2007; Lage et al., 2008; Yin et al., 2003). Recent studies have indicated that AMPK may be critical in promoting energy dissipation within adipocytes (Gaidhu et al., 2009). Although the kinase(s) and physiological consequence of phosphorylation are unknown, mass spectrometry analysis identified two phosphorylated serine residues in murine desnutrin (S406 and S430). Upon examination of those sites, S406 was found to be a perfect AMPK consensus site (FIG. 3J). To test if desnutrin is phosphorylated by AMPK an in vitro kinase assay was performed using purified AMPK, [γ-³²P]ATP; and desnutrin was immunoprecipitated from HEK293 cells transfected with desnutrin. Indeed, desnutrin was found to be phosphorylated by AMPK (FIG. 3K). To determine the specific site(s) that AMPK phosphorylates mutant forms of desnutrin at two known phosphorylation sites (S406A and S430A) were generated, and the in vitro kinase assay was performed. It was found that while wild type, as well as the S430A desnutrin mutant, were phosphorylated by AMPK, the S406A mutant was not, indicating S406 of desnutrin to be a unique and bonafide AMPK phosphorylation site (FIG. 3L). Interestingly, the amino acid sequence at S406 of desnutrin is also a perfect 14-3-3 binding motif. Using a 14-3-3 phospho-binding peptide antibody, it was found that desnutrin was recognized by this phospho-antibody, while the S406A desnutrin mutant was not (FIG. 3K). Immunoprecipitation of HEK 293 cells co-transfected with HA-tagged desnutrin-GFP or GFP control and Myc-tagged 14-3-3 showed interaction between desnutrin and 14-3-3. Interaction of endogenous proteins was detected by co-immunoprecipitation of desnutrin and 14-3-3 using WAT lysates. Furthermore, by using GST-14-3-3 and in vitro translated desnutrin, a direct interaction between desnutrin and 14-3-3 was detected.

The role of AMPK in lipolysis is controversial, with several reporting AMPK stimulates lipolysis and others showing it inhibits lipolysis, via phosphorylation of HSL at S563 (Daval et al., 2005; Koh et al., 2007; Lage et al., 2008; Yin et al., 2003). To determine the effect of AMPK specifically on desnutrin-mediated Lipolysis, oleate loaded HEK 293 cells, transfected with wild type desnutrin-HA-GFP, mutant S406A-desnutrin-HA-GFP or GFP control, were treated with the cell-permeable AMPK-activator, 5-amino-4-imidazolecarboxamide riboside (AICAR), and lipolysis was determined by measuring glycerol release. It was found that AICAR increased glycerol release by 1.8-fold from wild type desnutrin-HA-GFP transfected cells but failed to do so in S406A-desnutrin-HA-GFP transfected cells, indicating that AMPK-activation increases lipolysis via phosphorylation of S406A of desnutrin (FIG. 3M). To test the effect of AMPK on lipolysis in vivo, AICAR was administered intraperitoneally to flox/flox and desnutrin-ASKO mice and then measured serum FA levels. Five hours after injection, AICAR increased serum FA levels in flox/flox mice, however serum FA levels were unchanged in vehicle treated as well as AICAR treated desnutrin-ASKO mice indicating the AMPK-mediated increase in lipolysis is desnutrin-dependent (FIG. 3N). Therefore, AMPK phosphorylates desnutrin to increase lipolysis and promote FA oxidation in adipocytes.

Desnutrin-ASKO Mice have Improved Insulin Sensitivity and Decreased Ectopic TAG Storage

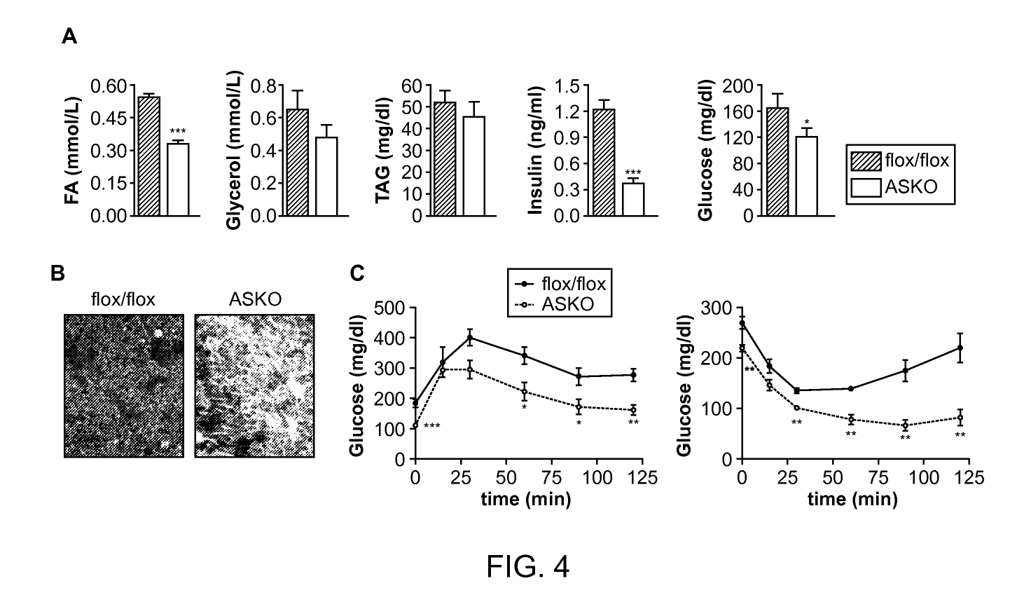

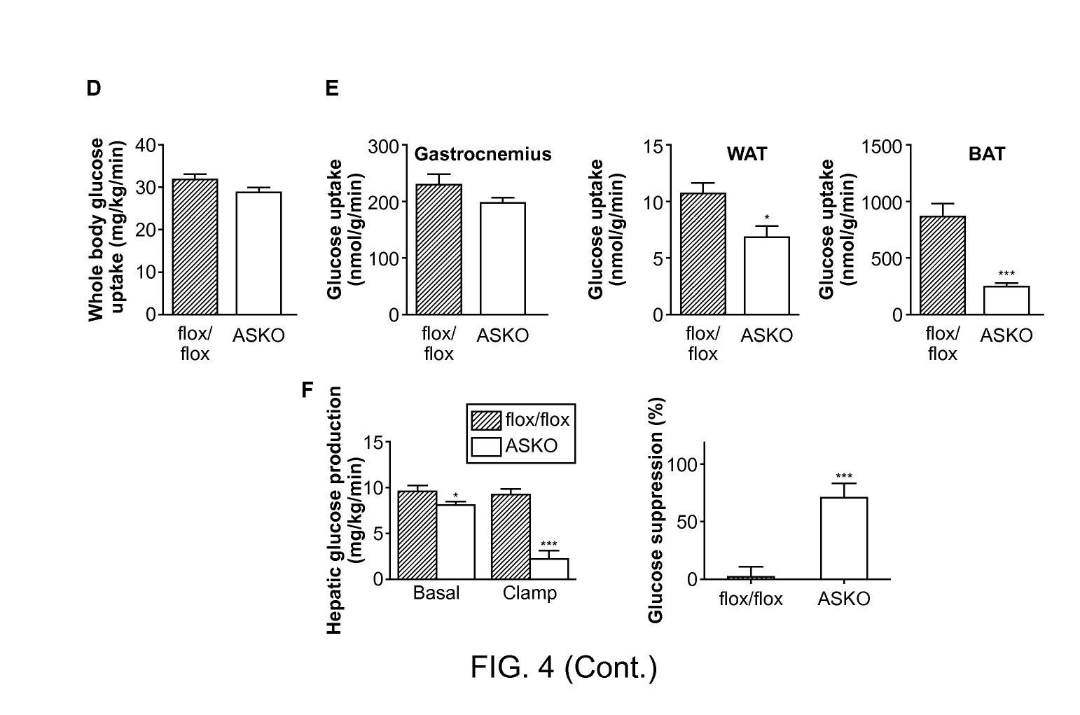

Desnutrin-ASKO mice exhibit impaired lipolysis and increased adiposity. Since adiposity is positively correlated with insulin resistance, it was postulated that these mice might be more insulin resistant. On the other hand, since FAs are know to exert lipotoxic effects that disrupt insulin signaling, the impaired lipolysis in desnutrin-ASKO mice may protect these mice from high-fat-diet induced insulin resistance (Samuel et al.). Consistent with blunted adipocyte lipolysis, serum FA levels were decreased by 39% in desnutrin-ASKO mice (FIG. 4A). Furthermore, fasting levels of glucose and insulin were both decreased in desnutrin-ASKO mice fed a HFD (FIG. 4A). While no difference in lipid staining with Oil Red O in skeletal muscle was found, there was less staining in the liver of desnutrin-ASKO mice, revealing decreased ectopic TAG storage, potentially due to lower circulating FA levels (FIG. 4B). Supporting this finding, liver weight was decreased by 32% in desnutrin-ASKO mice fed a HFD (FIG. 1E). Glucose and insulin tolerance tests (GTT and ITT) were performed on flox/flox and desnutrin-ASKO mice. Desnutrin-ASKO mice showed improved glucose clearance during a GTT (FIG. 4C, left). During an ITT, desnutrin-ASKO mice exhibited a prolonged response to insulin compared to flox/flox mice (FIG. 4C, right).

To gain further insight into the improved insulin sensitivity and to discern the impact of desnutrin ablation on peripheral and hepatic insulin action, a hyperinsulinemic-euglycemic clamp with radioisotope-labeled glucose infusion was performed on HFD-fed flox/flox and desnutrin-ASKO mice (Figure S5). The steady state glucose infusion rate during the clamps and whole body glucose uptake were unchanged in desnutrin-ASKO mice (FIG. 4D). Consistent with these findings, skeletal muscle 2-deoxyglucose (2-DOG) uptake was also not different between the two groups of mice (FIG. 4E, left). Notably, 2-DOG uptake in WAT and BAT were decreased on a per gram basis, although the substantial increase in adipose tissue mass likely made total uptake in desnutrin-ASKO WAT and BAT higher, consistent with the finding of no net change in whole body glucose uptake (FIGS. 4D and E, middle and right). However, hepatic insulin sensitivity was markedly improved in desnutrin-ASKO mice. Hepatic glucose production was 16% lower under basal conditions and 76% lower during the clamp (FIG. 4F, left). The ability to suppress hepatic glucose production was 37-fold higher in desnutrin-ASKO mice (FIG. 4F, right), consistent with the findings of decreased ectopic TAG storage in the liver. Taken together, these finding indicate that impaired adipocyte lipolysis in desnutrin-ASKO mice led to decreased circulating FA levels preventing ectopic TAG storage in the liver and improving hepatic insulin sensitivity. Increased adiposity and decreased FA oxidation in adipose tissue do not appear to contribute to insulin sensitivity. Rather decreased serum FA levels appear to be the major factor in improving insulin sensitivity in these mice.

FIGS. 1A-G. Increased adiposity in desnturin-ASKO mice. A) Western blot analysis from 40 μg of lysates from WAT, BAT, heart and liver from flox/flox and desnutrin-ASKO mice, using a desnutrin-specific antibody (upper) and RT-qPCR for desnutrin expression in the macrophage and BAT of flox/flox and desnutrin-ASKO mice (lower). B) Representative photographs of male flox/flox and desnutrin-ASKO at 16-weeks of age on a HFD. C) Representative photographs of gonadal, renal and BAT fat depots (upper, middle and lower). D) Gonadal (Gon), subcutaneous (SQ), renal (Ren) and brown adipose tissue (BAT) fat pad weights and E) liver, kidney, heart and lung weight from 16 week-old HFD-fed male mice flox/flox and desnutrin-ASKO mice, expressed as a percent of body weight (n=7). F) Food intake expressed as a percent of body weight in flox/flox and desnutrin-ASKO mice. G) Hematoxylin & eosin (H&E)-stained paraffin-embedded sections of gonadal (upper) and BAT (lower) and quantification of cell size (right). Scale bar (WAT)=20 μM, scale bar (BAT)=40 μM. *P<0.05, **P<0.01, ***P<0.001.

FIGS. 2A-G. Decreased lipolysis in desnutrin-ASKO mice results in impaired thermogenesis and energy expenditure. A) Glycerol (left) and FA (right) release from 50 mg fresh explants of gonadal WAT of flox/flox and desnutrin-ASKO mice incubated under basal or stimulated with 10 μM isoproterenol. (n=6) B) Glycerol (upper) and FA (lower) release from isolated white adipocytes of incubated under basal conditions or stimulated conditions. C) FA release from explants of BAT from flox/flox or desnutrin-ASKO mice incubated under basal conditions. (n=3) D) Percent TAG turnover (left) and percent de novo palmitate turnover (right) in gonadal WAT and BAT from 20-week old female HFD-fed mice. E) Body temperatures of overnight-fasted flox/flox and desnutrin-ASKO mice exposed to the cold. F) Oxygen consumption rate (VO2) measured through indirect calorimetry. G) Oxygen consumption rate (VO2) measured through indirect calorimetry after intraperitoneal injection of CL316243. (n=6) *P<0.05, **P<0.01, ***P<0.001.

FIGS. 3A-N. Desnutrin ablation converts BAT to WAT, and phosphorylation of desnutrin by AMPK increases lipolysis. A) Transmission electron microscopy from BAT of flox/flox and desnutrin-ASKO mice at 20-weeks of age showing the lipid droplet, scale bar=2 μM, or B) focusing in on mitochondria, scale bar=2 μM, C) RT-qPCR for the expression of genes involved in brown adipocyte differentiation. D) RT-qPCR for the expression of genes involved in brown adipocyte function (left), transcriptional co-repressors (middle) and white adipose-specific genes (right) from BAT of flox/flox and desnutrin-ASKO mice. (n=5-10). E) RT-qPCR for the expression of brown adipose-specific genes from WAT of flox/flox and desnutrin-ASKO mice. (n=5-10) F) Western blotting (upper) and immunostaining (lower) for UCP-1 from BAT of flox/flox and desnutrin-ASKO mice. G) RT-qPCR for PPARα, δ and γ from WAT and BAT of wild type mice (n=3-5). H) Chromatin immunoprecipitation (ChIP) using a PPARα, RIP140 or control GAPDH antibody to determine binding to the UCP-1 promoter. I) FA oxidation, measured by ¹⁴CO₂ production from [U¹⁴C] palmitate, from isolated brown adipocytes (left) and white adipocytes (right) from flox/flox and desnutrin-ASKO mice (n=4). J) AMPK consensus motif and murine desnutrin S406. J) Audioradiography to detect phosphorylated desnutrin after an in vitro kinase assay using [γ-³²P] ATP, purified AMPK and WT desnutrin and S406A desnutrin mutant immunoprecipitated from HEK 293 cells (top) and western blot using a phospho antibody to detect phosphorylation of S406 of desnutrin (middle) and using an anti-HA antibody to detect total desnutrin protein (lower) L) Audioradiography for phosphorylated desnutrin and western blot using an HA antibody for total desnutrin, after the same in vitro kinase assay, described above, but including S430A desnutrin mutant. M) Glycerol release from HEK 293 cells pre-loaded with oleic acid and transfected with WT desnutrin or S406 desnutrin mutant, treated with or without AICAR. Western blot showing transfection (inset). N) Serum FA levels from flox/flox or desnutrin-ASKO mice under basal conditions or after 5 hours of injection with AICAR or vehicle (n=5).

FIGS. 4A-F. Improved insulin sensitivity in desnutrin-ASKO mice. A) Serum parameters (n=6-8) B) Cryosections of frozen livers stained with Oil red O. Nuclei stained with hemotoxylin. C) Glucose and insulin tolerance tests (GTT and ITT) from 12-week old male mice fed a HFD (n=6) D) Whole body and E) tissue specific glucose uptake as well as F) hepatic glucose production (left) and percent glucose suppression (right) determined from hyperinsulinemic euglycemic clamping studies on flox/flox and desnutrin-ASKO mice. *P<0.05, **P<0.01, ***P<0.001.

### REFERENCES

Ahmadian, M., Duncan, R. E., and Sul, H. S. (2009a). The skinny on fat: lipolysis and fatty acid utilization in adipocytes. Trends Endocrinol Metab 20, 424-428.
Ahmadian, M., Duncan, R. E., Varady, K. A., Frasson, D., Hellerstein, M. K., Birkenfeld, A. L., Samuel, V. T., Shulman, G. I., Wang, Y., Kang, C., and Sul, H. S. (2009b). Adipose overexpression of desnutrin promotes fatty acid use and attenuates diet-induced obesity. Diabetes 58, 855-866.
Ahmadian, M., Wang, Y., and Sul, H. S. Lipolysis in adipocytes. Int J Biochem Cell Biol 42, 555-559.
Bachman, E. S., Dhillon, H., Zhang, C. Y., Cinti, S., Bianco, A. C., Kobilka, B. K., and Lowell, B. B. (2002). betaAR signaling required for diet-induced thermogenesis and obesity resistance. Science 297, 843-845.
Barbatelli, G., Murano, I., Madsen, L., Hao, Q., Jimenez, M., Kristiansen, K., Giacobino, J. P., De Matteis, R., and Cinti, S. The emergence of cold-induced brown adipocytes in mouse white fat depots is determined predominantly by white to brown adipocyte transdifferentiation. Am J Physiol Endocrinol Metab 298, E1244-1253.
Barbera, M. J., Schluter, A., Pedraza, N., Iglesias, R., Villarroya, F., and Giralt, M. (2001). Peroxisome proliferator-activated receptor alpha activates transcription of the brown fat uncoupling protein-1 gene. A link between regulation of the thermogenic and lipid oxidation pathways in the brown fat cell. J Biol Chem 276, 1486-1493.
Cannon, B., and Nedergaard, J. (2004). Brown adipose tissue: function and physiological significance. Physiol Rev 84, 277-359.
Chakravarthy, M. V., Lodhi, U., Yin, L., Malapaka, R. R., Xu, H. E., Turk, J., and Semenkovich, C. F. (2009). Identification of a physiologically relevant endogenous ligand for PPARalpha in liver. Cell 138, 476-488.
Christian, M., Kiskinis, E., Debevec, D., Leonardsson, G., White, R., and Parker, M. G. (2005). RIP140-targeted repression of gene expression in adipocytes. Mol Cell Biol 25, 9383-9391.
Chung, C., Doll, J. A., Gattu, A. K., Shugrue, C., Cornwell, M., Fitchev, P., and Crawford, S. E. (2008). Anti-angiogenic pigment epithelium-derived factor regulates hepatocyte triglyceride content through adipose triglyceride lipase (ATGL). J Hepatol 48, 471-478.
Daval, M., Diot-Dupuy, F., Bazin, R., Hainault, I., Viollet, B., Vaulont, S., Hajduch, E., Ferre, P., and Foufelle, F. (2005). Anti-lipolytic action of AMP-activated protein kinase in rodent adipocytes. J Biol Chem 280, 25250-25257.
Duncan, R. E., Ahmadian, M., Jaworski, K., Sarkadi-Nagy, E., and Sul, H. S. (2007). Regulation of lipolysis in adipocytes. Annu Rev Nutr 27, 79-101.
Ellis, J. M., Li, L. O., Wu, P. C., Koves, T. R., Ilkayeva, O., Stevens, R. D., Watkins, S. M., Muoio, D. M., and Coleman, R. A. Adipose acyl-CoA synthetase-1 directs fatty acids toward beta-oxidation and is required for cold thermogenesis. Cell Metab 12, 53-64.
Evans, R. M., Barish, G. D., and Wang, Y. X. (2004). PPARs and the complex journey to obesity. Nat Med 10, 355-361.
Folch, J., Lees, M., and Sloane Stanley, G. H. (1957). A simple method for the isolation and purification of total lipides from animal tissues. J Biol Chem 226, 497-509.
Frontini, A., and Cinti, S. Distribution and development of brown adipocytes in the murine and human adipose organ. Cell Metab 11, 253-256.
Fruhbeck, G., Becerril, S., Sainz, N., Garrastachu, P., and Garcia-Velloso, M. J. (2009). BAT: a new target for human obesity? Trends Pharmacol Sci 30, 387-396.
Gaidhu, M. P., Fediuc, S., Anthony, N. M., So, M., Mirpourian, M., Perry, R. L., and Ceddia, R. B. (2009). Prolonged AICAR-induced AMP-kinase activation promotes energy dissipation in white adipocytes: novel mechanisms integrating HSL and ATGL. J Lipid Res 50, 704-715.
Gesta, S., Tseng, Y. H., and Kahn, C. R. (2007). Developmental origin of fat: tracking obesity to its source. Cell 131, 242-256.
Granneman, J. G., Moore, H. P., Krishnamoorthy, R., and Rathod, M. (2009). Perilipin controls lipolysis by regulating the interactions of AB-hydrolase containing 5 (Abhd5) and adipose triglyceride lipase (Atgl). J Biol Chem 284, 34538-34544.
Haemmerle, G., Lass, A., Zimmermann, R., Gorkiewicz, G., Meyer, C., Rozman, J., Heldmaier, G., Maier, R., Theussl, C., Eder, S., Kratky, D., Wagner, E. F., Klingenspor, M., Hoefler, G., and Zechner, R. (2006). Defective lipolysis and altered energy metabolism in mice lacking adipose triglyceride lipase. Science 312, 734-737.
Jaworski, K., Ahmadian, M., Duncan, R. E., Sarkadi-Nagy, E., Varady, K. A., Hellerstein, M. K., Lee, H.-Y., Samuel, V. T., Shulman, G. I., Kim, K.-H., de Val, S., Kang, C., and Sul, H. S. (2009). AdPLA ablation increases lipolysis and prevents obesity induced by high-fat feeding or leptin deficiency. Nat Med 15, 159-168.
Jenkins, C. M., Mancuso, D. J., Yan, W., Sims, H. F., Gibson, B., and Gross, R. W. (2004). Identification, cloning, expression, and purification of three novel human calcium-independent phospholipase A2 family members possessing triacylglycerol lipase and acylglycerol transacylase activities. J Biol Chem 279, 48968-48975.
Kahn, B. B., Alquier, T., Carling, D., and Hardie, D. G. (2005). AMP-activated protein kinase: ancient energy gauge provides clues to modern understanding of metabolism. Cell Metab 1, 15-25.
Kienesberger, P. C., Lee, D., Pulinilkunnil, T., Brenner, D. S., Cai, L., Magnes, C., Koefeler, H. C., Streith, I. E., Rechberger, G. N., Haemmerle, G., Flier, J. S., Zechner, R., Kim, Y. B., and Kershaw, E. E. (2009). Adipose triglyceride lipase deficiency causes tissue-specific changes in insulin signaling. J Biol Chem 284, 30218-30229.
Kim, S. C., Chen, Y., Mirza, S., Xu, Y., Lee, J., Liu, P., and Zhao, Y. (2006). A clean, more efficient method for in-solution digestion of protein mixtures without detergent or urea. J Proteome Res 5, 3446-3452.
Koh, H. J., Hirshman, M. F., He, H., Li, Y., Manabe, Y., Balschi, J. A., and Goodyear, L. J. (2007). Adrenaline is a critical mediator of acute exercise-induced AMP-activated protein kinase activation in adipocytes. Biochem J 403, 473-481.
Lage, R., Dieguez, C., Vidal-Puig, A., and Lopez, M. (2008). AMPK: a metabolic gauge regulating whole-body energy homeostasis. Trends Mol Med 14, 539-549.
Latasa, M. J., Griffin, M. J., Moon, Y. S., Kang, C., and Sul, H. S. (2003). Occupancy and function of the −150 sterol regulatory element and −65 E-box in nutritional regulation of the fatty acid synthase gene in living animals. Mol Cell Biol 23, 5896-5907.
Leonardsson, G., Steel, J. H., Christian, M., Pocock, V., Milligan, S., Bell, J., So, P. W., Medina-Gomez, G., Vidal-Puig, A., White, R., and Parker, M. G. (2004). Nuclear receptor corepressor RIP140 regulates fat accumulation. Proc Natl Acad Sci USA 101, 8437-8442.
Mandard, S., Zandbergen, F., Tan, N. S., Escher, P., Patsouris, D., Koenig, W., Kleemann, R., Bakker, A., Veenman, F., Wahli, W., Muller, M., and Kersten, S. (2004). The direct peroxisome proliferator-activated receptor target fasting-induced adipose factor (FIAF/PGAR/ANGPTL4) is present in blood plasma as a truncated protein that is increased by fenofibrate treatment. J Biol Chem 279, 34411-34420.
Martinez-Agustin, O., Hernandez-Morante, J. J., Martinez-Plata, E., Sanchez de Medina, F., and Garaulet, M. Differences in AMPK expression between subcutaneous and visceral adipose tissue in morbid obesity. Regul Pept 163, 31-36.
Mulligan, J. D., Gonzalez, A. A., Stewart, A. M., Carey, H. V., and Saupe, K. W. (2007). Upregulation of AMPK during cold exposure occurs via distinct mechanisms in brown and white adipose tissue of the mouse. J Physiol 580, 677-684.
Narbonne, P., and Roy, R. (2009). Caenorhabditis elegans dauers need LKB1/AMPK to ration lipid reserves and ensure long-term survival. Nature 457, 210-214.
Narkar, V. A., Downes, M., Yu, R. T., Embler, E., Wang, Y. X., Banayo, E., Mihaylova, M. M., Nelson, M. C., Zou, Y., Juguilon, H., Kang, H., Shaw, R. J., and Evans, R. M. (2008). AMPK and PPARdelta agonists are exercise mimetics. Cell 134, 405-415.
Patsouris, D., Mandard, S., Voshol, P. J., Escher, P., Tan, N. S., Havekes, L. M., Koenig, W., Marz, W., Tafuri, S., Wahli, W., Muller, M., and Kersten, S. (2004). PPARalpha governs glycerol metabolism. J Clin Invest 114, 94-103.
Petrovic, N., Walden, T. B., Shabalina, I. G., Timmons, J. A., Cannon, B., and Nedergaard, J. Chronic peroxisome proliferator-activated receptor gamma (PPARgamma) activation of epididymally derived white adipocyte cultures reveals a population of thermogenically competent, UCP1-containing adipocytes molecularly distinct from classic brown adipocytes. J Biol Chem 285, 7153-7164.
Reid, B. N., Ables, G. P., Otlivanchik, O. A., Schoiswohl, G., Zechner, R., Blaner, W. S., Goldberg, U., Schwabe, R. F., Chua, S. C., Jr., and Huang, L. S. (2008). Hepatic overexpression of hormone-sensitive lipase and adipose triglyceride lipase promotes fatty acid oxidation, stimulates direct release of free fatty acids, and ameliorates steatosis. J Biol Chem 283, 13087-13099.
Samuel, V. T., Choi, C. S., Phillips, T. G., Romanelli, A. J., Geisler, J. G., Bhanot, S., McKay, R., Monia, B., Shutter, J. R., Lindberg, R. A., Shulman, G. I., and Veniant, M. M. (2006). Targeting foxol in mice using antisense oligonucleotide improves hepatic and peripheral insulin action. Diabetes 55, 2042-2050.
Samuel, V. T., Petersen, K. F., and Shulman, G. I. Lipid-induced insulin resistance: unravelling the mechanism. Lancet 375, 2267-2277.
Seale, P., Bjork, B., Yang, W., Kajimura, S., Chin, S., Kuang, S., Scime, A., Devarakonda, S., Conroe, H. M., Erdjument-Bromage, H., Tempst, P., Rudnicki, M. A., Beier, D. R., and Spiegelman, B. M. (2008). PRDM16 controls a brown fat/skeletal muscle switch. Nature 454, 961-967.
Turner, S. M., Murphy, E. J., Neese, R. A., Antelo, F., Thomas, T., Agarwal, A., Go, C., and Hellerstein, M. K. (2003). Measurement of TG synthesis and turnover in vivo by 2H2O incorporation into the glycerol moiety and application of MIDA. Am J Physiol Endocrinol Metab 285, E790-803.
van der Meer, D. L., Degenhardt, T., Vaisanen, S., de Groot, P. J., Heinaniemi, M., de Vries, S. C., Muller, M., Carlberg, C., and Kersten, S. Profiling of promoter occupancy by PPARalpha in human hepatoma cells via ChIP-chip analysis. Nucleic Acids Res 38, 2839-2850.
Villena, J. A., Roy, S., Sarkadi-Nagy, E., Kim, K. H., and Sul, H. S. (2004a). Desnutrin, an adipocyte gene encoding a novel patatin domain-containing protein, is induced by fasting and glucocorticoids: ectopic expression of desnutrin increases triglyceride hydrolysis. J Biol Chem 279, 47066-47075.
Villena, J. A., Viollet, B., Andreelli, F., Kahn, A., Vaulont, S., and Sul, H. S. (2004b). Induced adiposity and adipocyte hypertrophy in mice lacking the AMP-activated protein kinase-alpha2 subunit. Diabetes 53, 2242-2249.
Wong, R. H., Chang, I., Hudak, C. S., Hyun, S., Kwan, H. Y., and Sul, H. S. (2009). A role of DNA-PK for the metabolic gene regulation in response to insulin. Cell 136, 1056-1072.
Yaffe, M. B., Rittinger, K., Volinia, S., Caron, P. R., Aitken, A., Leffers, H., Gamblin, S. J., Smerdon, S. J., and Cantley, L. C. (1997). The structural basis for 14-3-3:phosphopeptide binding specificity. Cell 91, 961-971.
Yang, X., Lu, X., Lombes, M., Rha, G. B., Chi, Y. I., Guerin, T. M., Smart, E. J., and Liu, J. The G(0)/G(1) switch gene 2 regulates adipose lipolysis through association with adipose triglyceride lipase. Cell Metab 11, 194-205.
Yin, W., Mu, J., and Birnbaum, M. J. (2003). Role of AMP-activated protein kinase in cyclic AMP-dependent lipolysis In 3T3-L1 adipocytes. J Biol Chem 278, 43074-43080.
Zimmermann, R., Strauss, J. G., Haemmerle, G., Schoiswohl, G., Birner-Gruenberger, R., Riederer, M., Lass, A., Neuberger, G., Eisenhaber, F., Hermetter, A., and Zechner, R. (2004). Fat mobilization in adipose tissue is promoted by adipose triglyceride lipase. Science 306, 1383-1386.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of converting white adipose tissue (WAT) to brown adipose tissue (BAT), the method comprising contacting WAT adipocytes with a desnutrin polypeptide, a desnutrin nucleic acid comprising a nucleotide sequence encoding a desnutrin polypeptide, or an agent that activates 5′-adenosine monophosphate kinase, wherein said contacting results in an increase in the level and/or activity of desnutrin in the WAT adipocytes, and conversion of the WAT adipocytes to BAT adipocytes.

2. The method of claim 1, wherein at least about 5% of the WAT adipocytes are converted to BAT adipocytes.

3. The method of claim 1, wherein said contacting increases the level of at least one BAT-selective gene product in the WAT adipocyte.

4. The method of claim 1, wherein said contacting increases uncoupling or fatty acid oxidation in the WAT adipocyte.

5. The method of claim 1, comprising contacting WAT adipocytes with a desnutrin polypeptide.

6. The method of claim 1, comprising contacting WAT adipocytes with a desnutrin nucleic acid.

7. The method of claim 6, wherein the desnutrin nucleic acid is a recombinant viral vector.

8. A method of treating obesity in an individual, the method comprising administering to the individual an effective amount of a desnutrin polypeptide or a desnutrin nucleic acid comprising a nucleotide sequence encoding a desnutrin polypeptide, wherein said administering converts white adipose tissue to brown adipose tissue in the individual.

9. The method of claim 8, wherein the individual has a body mass index greater than 25 kg/m².

10. The method of claim 8, comprising administering a desnutrin polypeptide.

11. The method of claim 10, wherein the desnutrin polypeptide is formulated with a biodegradable hydrogel or a biodegradable microparticle.

12. The method of claim 8, comprising administering a desnutrin nucleic acid.

13. The method of claim 12, wherein the desnutrin nucleic acid is a recombinant viral vector.

14. An in vitro method for identifying an agent that increases desnutrin activity and/or levels, the method comprising:
a) contacting desnutrin with a test agent in the presence of a desnutrin substrate; and
b) determining the effect, if any, of the test agent on desnutrin activity and/or levels, wherein an agent that increases desnutrin activity and/or levels is considered a candidate agent for converting white adipose tissue to brown adipose tissue.

15. The method of claim 14, wherein the desnutrin substrate is a triacylglyceride (TAG).

16. The method of claim 14, wherein the TAG comprises a detectably labeled fatty acid, and wherein said determining step comprises detecting labeled free fatty acid released from the TAG by the desnutrin.

17. The method of claim 14, wherein the assay is a cell-free assay.

18. The method of claim 14, wherein the assay is a cell-based assay.

19. The method of claim 18, wherein the desnutrin is produced in a host cell that has been genetically modified with a recombinant expression vector comprising a nucleotide sequence encoding the desnutrin.

20. The method of claim 14, wherein an agent that increases desnutrin activity and/or levels is considered a candidate agent for treating obesity.
